(19) United States
(12) Patent Application Publication
(10) Pub. No.: US 2025/0375233 A1
(43) Pub. Date: Dec. 11, 2025

(54) SURGICAL INSTRUMENT

(71) Applicant: LIVSMED INC., Seongnam-si (KR)

(72) Inventor: Dong Hoon KANG, Seoul (KR)

(21) Appl. No.: 19/229,678

(22) Filed: Jun. 5, 2025

(30) Foreign Application Priority Data
Jun. 7, 2024 (KR) 10-2024-0074252

(51) Int. Cl. A61B 18/00

(52) U.S. Cl. CPC A61B 18/00; A61B 2018/00077; A61B 2018/00083; A61B 2018/00178; A61B 2018/00184; A61B 2018/0091

(57) ABSTRACT

Provided is a surgical instrument including an end tool rotatable in at least one direction and including a first jaw and a second jaw opposite to the first jaw, a manipulation part configured to control a rotational motion of the end tool, connected to an external power supply, and further configured to transmit an electrical energy provided by the external power supply to the end tool, and a connection part configured to connect the end tool to the manipulation part, wherein the end tool further includes a coupling hub connected to one end of the connection part, and the first jaw includes a first jaw electrode part electrically connected to the external power supply and configured to receive the electrical energy, and a first jaw insulating part disposed between the first jaw electrode part and the coupling hub and configured to insulate the first jaw electrode part.

### CROSS-REFERENCE TO RELATED APPLICATION

This application is based on and claims priority under 35 USC § 119 to Korean Patent Application No. 10-2024-0074252, filed on Jun. 7, 2024, in the Korean Intellectual Property Office, the disclosure of which is incorporated by reference herein in its entirety.

### BACKGROUND

### 1. Field

The present disclosure relates to a surgical instrument, and more particularly, to a surgical instrument that is mountable on a robot arm or operable manually for use in laparoscopic surgery or various other surgical procedures.

### 2. Description of the Related Art

In many cases, surgical procedures require the cutting and joining of body tissues, including organs, muscle tissue, connective tissue, and blood vessels. Over the centuries, sharp blades and sutures have been used for cutting and joining. However, during surgical procedures, cutting body tissues, especially those that are highly vascularized, results in bleeding. Accordingly, surgeons have sought surgical instruments and methods that can slow down or reduce bleeding during surgical procedures.

Recently, it has become possible to use electrosurgical instruments that use electrical energy for performing specific types of surgical tasks. For example, electrosurgical instruments equipped with one or more electrodes configured to supply electrical energy have been developed for use with surgical tools such as graspers, scissors, forceps, blades, needles, and hooks. Electrical energy supplied through the electrodes can be used to coagulate, join, or cut the patient's body tissues.

In particular, when using electrical energy, it becomes possible to perform cutting and hemostasis simultaneously.

Electrosurgical instruments are typically divided into two types, monopolar and bipolar. In the monopolar electrosurgical instrument, electrical energy of a specific polarity is supplied to one or more electrodes of the instrument. In addition, electrical energy of the opposite polarity is electrically connected to a patient. On the other hand, in the bipolar electrosurgical instrument, one or more electrodes are electrically connected to a first polarity electrical energy source, while one or more other electrodes are electrically connected to a second polarity electrical energy source opposite to the first polarity electrical energy source.

The background art described above is technical information retained by the present inventors in order to derive the present disclosure or obtained by the present inventors in the process of deriving the present disclosure, and thus is not necessarily known art disclosed to the general public before the filing of the present application.

### SUMMARY

The present disclosure is directed to providing a surgical instrument that is mountable on a robot arm or operable manually for use in laparoscopic surgery or various other surgical procedures, wherein the surgical instrument includes an end tool that is at least partially formed of a conductive material and configured to receive electrical energy from an external power supply to form an electrode.

According to an aspect of the present disclosure, there is provided is a surgical instrument including an end tool rotatable in at least one direction and including a first jaw and a second jaw opposite to the first jaw, a manipulation part configured to control a rotational motion of the end tool, connected to an external power supply, and further configured to transmit an electrical energy provided by the external power supply to the end tool, and a connection part configured to connect the end tool to the manipulation part, wherein the end tool further includes a coupling hub connected to one end of the connection part, and the first jaw includes a first jaw electrode part electrically connected to the external power supply and configured to receive the electrical energy, and a first jaw insulating part disposed between the first jaw electrode part and the coupling hub and configured to insulate the first jaw electrode part.

In an embodiment of the present disclosure, the coupling hub may be electrically connected to the external power supply and may receive the electrical energy, and the second jaw may receive the electrical energy through the coupling hub.

In an embodiment of the present disclosure, at least a portion of the second jaw, which is in contact with the coupling hub, may be formed of a conductive material.

In an embodiment of the present disclosure, the coupling hub may include an end tool hub coupled to one end of the end tool, and a pitch hub configured to connect the end tool hub to the connection part and disposed to at least partially overlap the end tool hub, wherein at least one of the end tool hub and the pitch hub comprises conductive material.

In an embodiment of the present disclosure, the end tool may further include a plurality of pulleys disposed on the coupling hub and configured to transmit power for rotating the end tool, wherein some of the plurality of pulleys include a conductive material and are electrically connected to the external power supply.

In an embodiment of the present disclosure, at least one of the plurality of pulleys may guide, to the first jaw electrode part, a first electric wire which is electrically connected to the external power supply and through which the electrical energy is transmitted to the first jaw electrode part.

In an embodiment of the present disclosure, the second jaw may include a second jaw electrode part electrically connected to the external power supply and configured to receive the electrical energy, and a second jaw insulating part disposed between the second jaw electrode part and the coupling hub and configured to insulate the second jaw electrode part.

In an embodiment of the present disclosure, the coupling hub may be formed of an insulating material.

In an embodiment of the present disclosure, the first jaw electrode part and the first jaw insulating part may be formed using a double injection molding method, and the second jaw electrode part and the second jaw insulating part may be formed using the double injection molding method.

In an embodiment of the present disclosure, the end tool may further include a plurality of pulleys disposed on the coupling hub and configured to transmit power for rotating the end tool, wherein at least one of the plurality of pulleys may guide, to the first jaw electrode part, a first electric wire which is electrically connected to the external power supply and through which the electrical energy is transmitted to the first jaw electrode part, and guide, to the second jaw electrode part, a second electric wire which is electrically connected to the external power supply and through which the electrical energy is transmitted to the second jaw electrode part.

According to another aspect of the present disclosure, there is provided is a surgical instrument including an end tool rotatable in at least one direction and including a first jaw and a second jaw opposite to the first jaw, a manipulation part configured to control a rotational motion of the end tool, connected to an external power supply, and further configured to transmit an electrical energy provided by the external power supply to the end tool, a connection part configured to connect the end tool to the manipulation part, and a power transmission part connected to the manipulation part and including a jaw wire configured to transmit a rotation of the manipulation part to the end tool, wherein the end tool may further include a coupling hub connected to one end of the connection part, and the jaw wire may include a first jaw wire positioned on the coupling hub and electrically connected to the first jaw, and a second jaw wire positioned on the coupling hub and electrically connected to the second jaw.

In an embodiment of the present disclosure, the first jaw may include a first wire-contact part that is coupled to the coupling hub and in contact with a portion of a first electric wire part of the first jaw wire, and the second jaw may include a second wire-contact part that is coupled to the coupling hub and in contact with a portion of a second electric wire part of the second jaw wire.

In an embodiment of the present disclosure, the end tool may further include an insulating sleeve disposed between the first wire-contact part and the second wire-contact part, and formed of an insulating material.

In an embodiment of the present disclosure, the coupling hub may be formed of an insulating material.

In an embodiment of the present disclosure, the jaw wire may include an electric wire part connected to the external power supply and formed of a conductive material, and a cover part surrounding the electric wire part and formed of an insulating material.

In an embodiment of the present disclosure, the cover part may be formed as a sheath surrounding the electric wire part.

In an embodiment of the present disclosure, the cover part may be formed as a tube coupled to the electric wire part.

Other aspects, features, and advantages other than those described above will become apparent from the following drawings, claims, and detailed description of the disclosure.

### BRIEF DESCRIPTION OF THE DRAWINGS

The above and other aspects, features, and advantages of certain embodiments of the disclosure will be more apparent from the following description taken in conjunction with the accompanying drawings in which:

FIGS. 1A-1D are views illustrating an example of use of a surgical instrument according to an embodiment of the present disclosure;

FIG. 2 is a perspective view illustrating a surgical instrument according to an embodiment of the present disclosure;

FIG. 3 is a side view illustrating the surgical instrument of FIG. 2;

FIGS. 4 to 6 are perspective views illustrating an end tool of the surgical instrument according to an embodiment of the present disclosure;

FIG. 7 is a plan view of the end tool of the surgical instrument of FIG. 4;

FIGS. 8A-8C are side views of the end tool of the surgical instrument of FIG. 4;

FIGS. 9A and 9B are views illustrating an open state of jaws of the end tool in the surgical instrument shown in FIG. 7;

FIGS. 10 and 11 are perspective views illustrating a manipulation part of the surgical instrument of FIG. 2;

FIG. 12 is a view schematically illustrating only a configuration of pulleys and wires constituting joints of the surgical instrument illustrated in FIG. 2;

FIG. 13 is a side view illustrating an electrode formation structure of an end tool according to an embodiment of the present disclosure;

FIG. 14 is a side view illustrating an electrode formation structure of an end tool according to another embodiment of the present disclosure;

FIGS. 15 to 17 are perspective views illustrating an end tool of a surgical instrument according to another embodiment of the present disclosure;

FIGS. 18A-18C are side views of the end tool of FIG. 15; and

FIG. 19 is a plan view illustrating an electrode formation structure of the end tool of FIG. 15.

### DETAILED DESCRIPTION

Hereinafter, the following embodiments will be described in detail with reference to the accompanying drawings. When describing with reference to the drawings, identical or corresponding components will be assigned the same reference numerals and duplicate descriptions thereof will be omitted.

Since various transformations can be made to these embodiments, specific embodiments will be illustrated in the drawings and described in detail in the detailed description. The effects and features of the present embodiments and the accompanying methods thereof will become apparent from the following description of the contents, taken in conjunction with the accompanying drawings. However, the present embodiments are not limited to the embodiments disclosed below, but may be implemented in various forms.

In describing the present disclosure, a detailed description of known related arts will be omitted when it is determined that the gist of the present disclosure may be unnecessarily obscured.

In the following embodiments, singular forms are intended to include plural forms as well, unless the context clearly indicates otherwise. Although terms such as “first,” “second,” and the like may be used to describe various components, such components should not be limited to the above terms The terms are only used to distinguish one component from another.

In the following embodiments, terms such as “include” or “have” means that the features or components described in the specification are present, and the possibility that one or more other features or components will be added is not excluded in advance.

In the following embodiments, when a unit, region, or component is referred to as being formed on another unit, region, or component, it can be directly formed on the other unit, region, or component. That is, for example, intervening units, regions, or components may be present.

In the following embodiments, terms such as “connecting” or “coupling” two members do not necessarily mean a direct and/or fixed connection or coupling of the two members, unless the context clearly indicates otherwise, and do not preclude another members from being interposed between the two members.

Sizes of components in the drawings may be exaggerated or reduced for convenience of description. For example, since the size and thickness of each component shown in the drawings are arbitrarily illustrated for convenience of description, the following embodiments are not necessarily limited thereto.

### Surgical Instrument

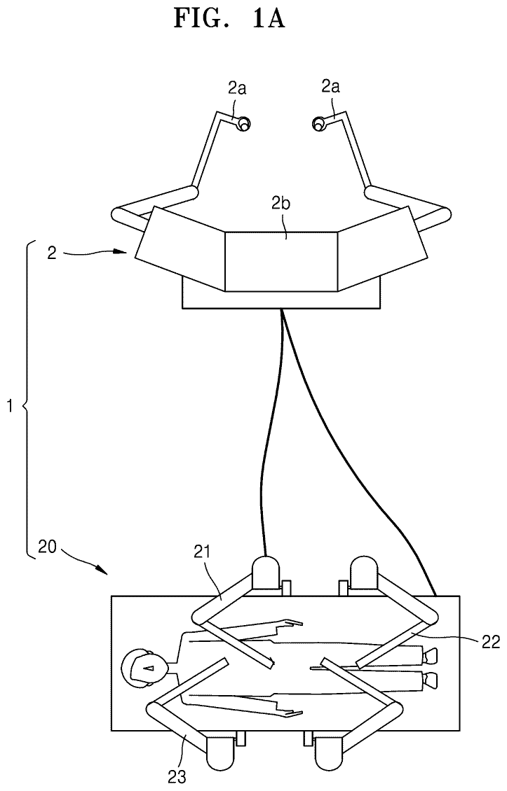

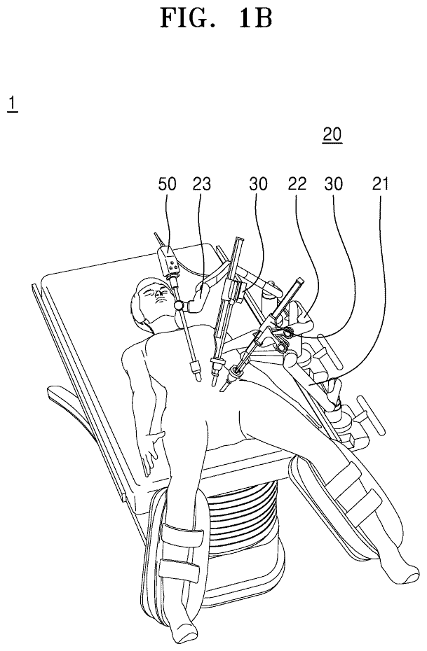

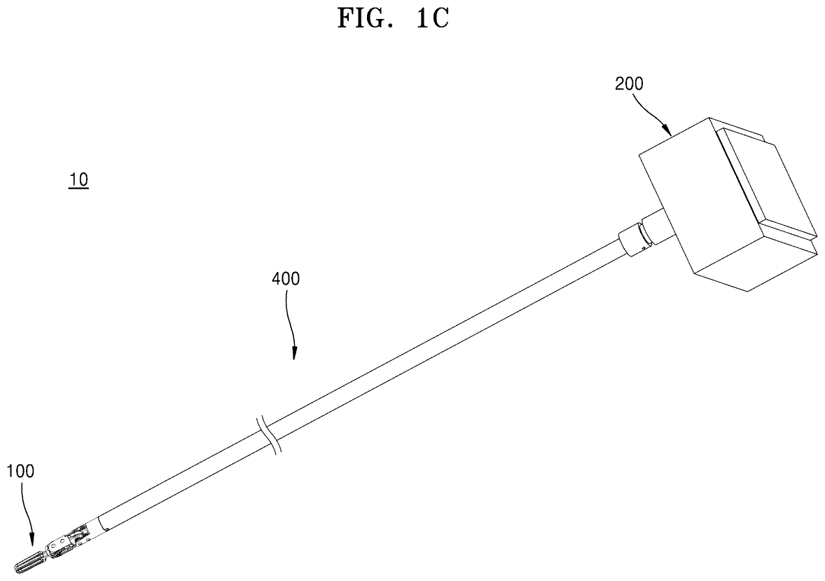

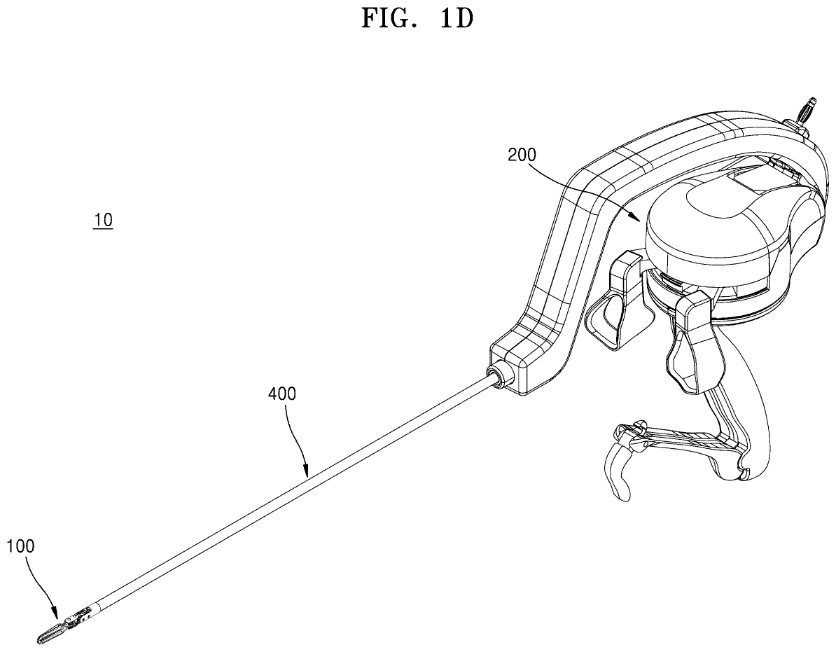

FIGS. 1A-1D are views illustrating an example of use of an end tool 100 of a surgical instrument 10 according to an embodiment of the present disclosure. Here, FIG. 1A is a conceptual view illustrating a surgical robot system 1 on which the end tool 100 of the surgical instrument 10 according to an embodiment of the present disclosure is mounted. FIG. 1B is a perspective view illustrating a slave robot 20 of the surgical robot system 1 of FIG. 1A, and FIG. 1C is a perspective view illustrating the surgical instrument 10 mounted on the slave robot 20 of FIG. 1A. In addition, FIG. 1D is a perspective view illustrating an example of the surgical instrument 10, which is of a hand-held type and on which the end tool 100 according to an embodiment of the present disclosure is mounted.

Referring to FIGS. 1A to 1C, the surgical robot system 1 includes a master robot 2, the slave robot 20, and the surgical instrument 10.

The master robot 2 includes manipulating members 2a and a display member 2b, and the slave robot 20 includes one or more robot arm units 21, 22, and 23.

In detail, the master robot 2 includes the manipulating members 2a so that a surgical operator can grip and manipulate them respectively with both hands. In addition, an image captured through a laparoscope 50 is displayed as a screen image on the display member 2b of the master robot 2. In addition, a predetermined virtual manipulation plate may be displayed independently or displayed together with the image captured by the laparoscope 50 on the display member 2b. A detailed description of the arrangement, configuration, and the like of such a virtual manipulation plate will be omitted.

Meanwhile, the slave robot 20 may include one or more robot arm units 21, 22, and 23. Here, each of the robot arm units 21, 22, and 23 may be provided in the form of a module that can operate independently of each other, and in this case, an algorithm for preventing a collision between the robot arm units 21, 22, and 23 may be applied to the surgical robot system 1.

Here, two or more of the robot arm units 21, 22, and 23 may each have the surgical instrument 10 attached thereto, and one or more of the robot arm units 21, 22, and 23 may have the laparoscope 50 attached thereto. In addition, the surgical operator may select the robot arm unit 21, 22, or 23 to be controlled via the master robot 2. As described above, by directly controlling a total of three or more surgical instruments through the master robot 2, the surgical operator may accurately and freely control various tools according to the intention of the surgical operator without a surgical assistant.

Continuing to refer to FIG. 1C, the surgical instrument 10 of the surgical robot system 1 may include the end tool 100, a manipulation part 200, and a connection part 400.

Here, the connection part 400 is formed in the shape of a hollow shaft, in which one or more wires may be accommodated, and may have one end portion to which the manipulation part 200 is coupled and another end portion to which the end tool 100 is coupled and serve to connect the manipulation part 200 to the end tool 100.

The manipulation part 200 is formed at one end portion of the connection part 400 and provides an interface capable of being coupled to the robot arm units 21, 22, and 23. Accordingly, when a user operates the master robot 2, a motor (not shown) of the robot arm units 21, 22, and 23 is operated so that the end tool 100 of the surgical instrument 10 can perform a motion corresponding thereto, and a driving force of the motor (not shown) is transmitted to the end tool 100 through the manipulation part 200. In other words, it may be described that the manipulation part 200 itself becomes an interface that connects between the surgical instrument 10 and the slave robot 20.

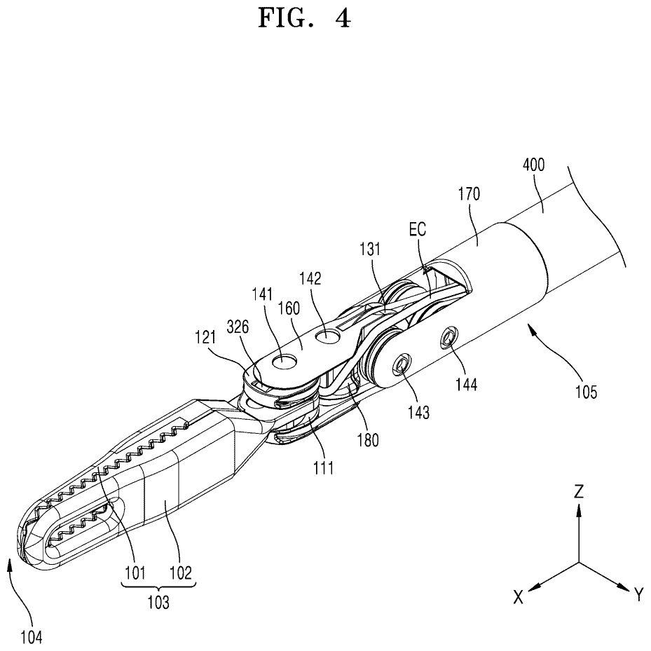

The end tool 100 is formed on another end portion of the connection part 400, and performs necessary motions for surgery by being inserted into a surgical site. The end tool 100 will be described in more detail later with reference to FIG. 4.

Meanwhile, referring to FIG. 1D, the hand-held type surgical instrument 10 may include the end tool 100, the manipulation part 200, and the connection part 400.

Here, the connection part 400 is formed in the shape of a hollow shaft, in which one or more wires (to be described later) may be accommodated, and may have one end portion to which the manipulation part 200 is coupled and another end portion to which the end tool 100 is coupled and serve to connect the manipulation part 200 to the end tool 100.

The manipulation part 200 is formed on one end portion of the connection part 400 and provided as an interface to be directly controlled by a medical doctor, for example, a tongs shape, a stick shape, a lever shape, or the like, and when the medical doctor controls the manipulation part 200, the end tool 100, which is connected to the interface and inserted into the body of a surgical patient, performs a certain motion, thereby performing surgery. Here, the manipulation part 200 is illustrated in FIG. 1D as being formed in a handle shape that is rotatable while the finger is inserted therein, the concept of the present disclosure is not limited thereto, and various types of manipulation parts 200 that are connected to the end tool 100 and manipulate the end tool 100 may be possible.

The end tool 100 is formed on another end portion of the connection part 400, and performs necessary motions for surgery by being inserted into a surgical site. The end tool 100 will be described in more detail later with reference to FIG. 4.

The end tool 100 of the surgical instrument according to an embodiment of the present disclosure may be provided in the surgical instrument 10 of the surgical robot system 1 shown in FIGS. 1A to 1C, or may also be provided in the hand-held type surgical instrument 10 shown in FIG. 1D.

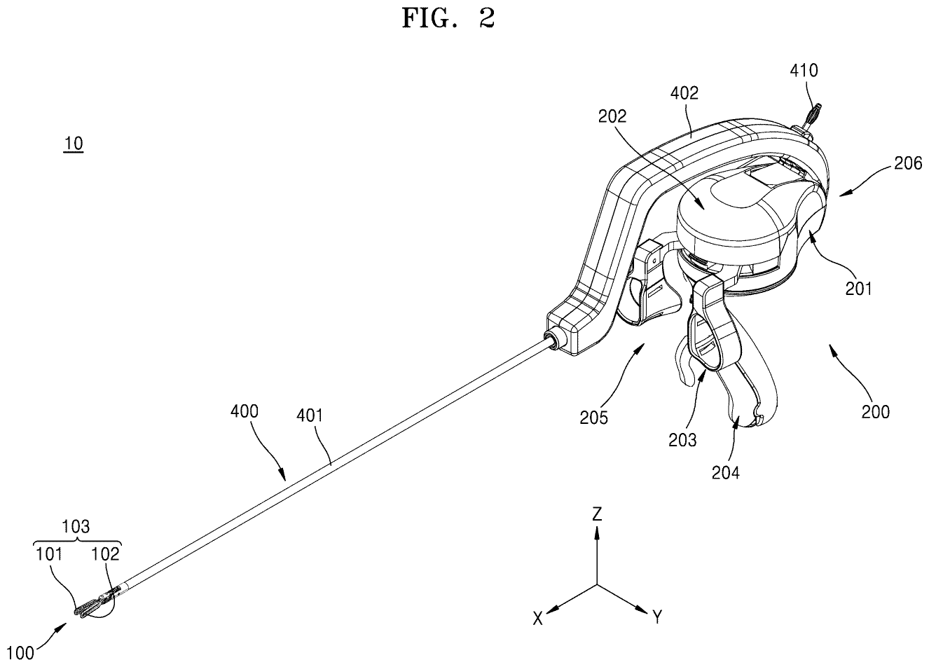

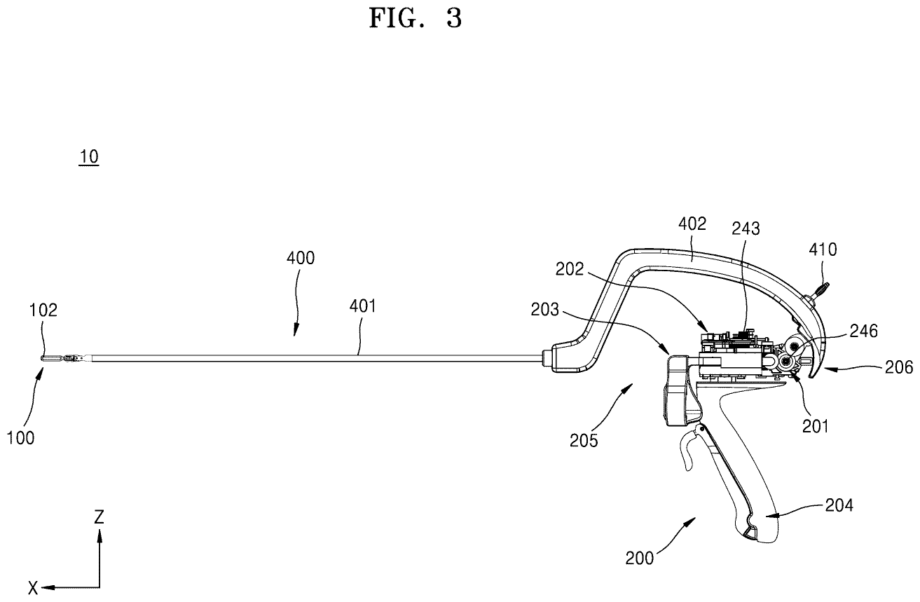

FIG. 2 is a perspective view illustrating a surgical instrument 10 according to an embodiment of the present disclosure, and FIG. 3 is a side view illustrating the surgical instrument 10 of FIG. 2.

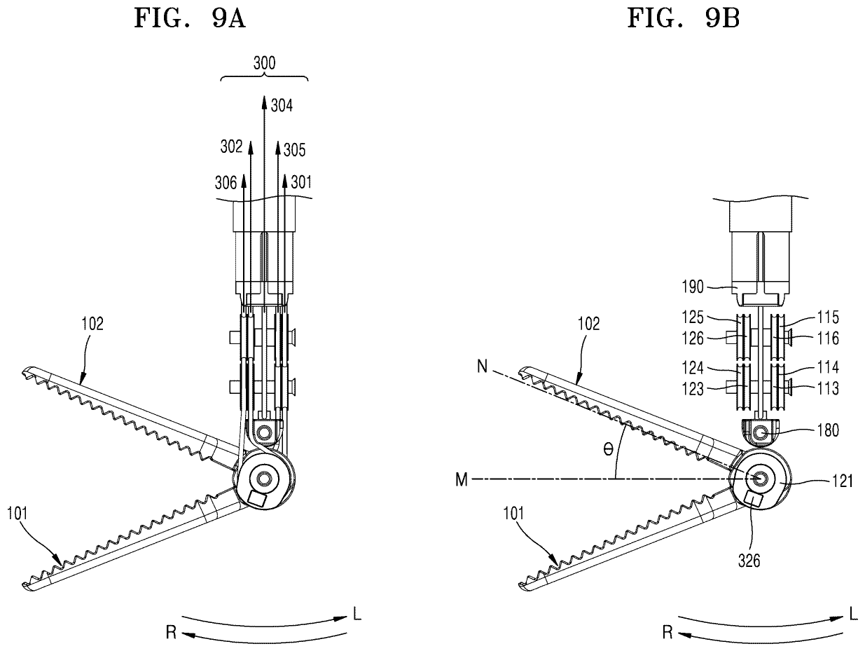

Referring to FIGS. 2 and 3, the surgical instrument 10 according to an embodiment of the present disclosure includes an end tool 100, a manipulation part 200, a power transmission part 300 (see FIGS. 9A and 9B), and a connection part 400.

Here, the connection part 400 is formed in the shape of a hollow shaft, and one or more wires and electric wires may be accommodated therein. The manipulation part 200 is coupled to one end portion of the connection part 400, the end tool 100 is coupled to another end portion thereof, and the connection part 400 may serve to connect the manipulation part 200 to the end tool 100. Here, the connection part 400 of the surgical instrument 10 according to an embodiment of the present disclosure includes a straight part 401 and a bent part 402, wherein the straight part 401 may be formed at a side coupled to the end tool 100, and the bent part 402 is formed at a side to which the manipulation part 200 is coupled. As such, since the end portion of the connection part 400 at the side of the manipulation part 200 is configured to be bent, a pitch manipulation part 201, a yaw manipulation part 202, and an actuation manipulation part 203 may be formed along an extension line of the end tool 100 or adjacent to the extension line. In other words, it may be said that the pitch manipulation part 201 and the yaw manipulation part 202 are at least partially accommodated in a concave portion formed by the bent part 402. Due to the above-described shape of the bent part 402, the shapes and motions of the manipulation part 200 and the end tool 100 may be further intuitively matched with each other.

Meanwhile, a plane on which the bent part 402 is formed may be substantially the same as a pitch plane, that is, an XZ plane of FIG. 2. As such, as the bent part 402 is formed on the plane substantially the same as the XZ plane, interference with the manipulation part 200 may be reduced. Of course, for intuitive motions of the end tool 100 and the manipulation part 200, any form other than the XZ plane may be possible.

Meanwhile, a connector 410 may be further formed on the bent part 402. The connector 410 may be connected to an external power source (not shown), and the connector 410 may be connected to a jaw 103 to transmit electrical energy supplied from the external power source (not shown) to the jaw 103. Here, the connector 410 may be of a bipolar type having two electrodes, or a monopolar type having one electrode.

The manipulation part 200 is formed on one end portion of the connection part 400 and provided as an interface to be directly controlled by a medical doctor, for example, a tongs shape, a stick shape, a lever shape, or the like, and when the medical doctor controls the manipulation part 200, the end tool 100, which is connected to the interface and inserted into the body of a surgical patient, performs a certain motion, thereby performing surgery. Here, the manipulation part 200 is illustrated in FIG. 2 as being formed in a handle shape that is rotatable while the finger is inserted therein, the concept of the present disclosure is not limited thereto, and various types of manipulation parts that are connected to the end tool 100 and manipulate the end tool 100 may be possible.

The end tool 100 is formed on another end portion of the connection part 400, and performs necessary motions for surgery by being inserted into a surgical site. In an example of the above-described end tool 100, as shown in FIG. 2, a pair of jaws 101 and 102 for performing a grip motion may be used. However, the concept of the present disclosure is not limited thereto, and various devices for performing surgery may be used as the end tool 100. For example, a configuration of a cantilever cautery may also be used as the end tool 100. The end tool 100 is connected to the manipulation part 200 by the power transmission part 300, and receives a driving force of the manipulation part 200 through the power transmission part 300 to perform a motion necessary for surgery, such as gripping, cutting, suturing, or the like.

Here, the end tool 100 of the surgical instrument 10 according to an embodiment of the present disclosure is configured to be rotatable in at least one or more directions. For example, the end tool 100 may be configured to perform a pitch motion around a Y-axis of FIG. 2 while simultaneously performing a yaw motion and an actuation motion around a Z-axis of FIG. 2.

Here, each of the pitch, yaw, and actuation motions used in the present disclosure are defined as follows.

First, the pitch motion means a motion of the end tool 100 rotating in a vertical direction with respect to an extension direction of the connection part 400 (an X-axis direction of FIG. 2), that is, a motion rotating around the Y-axis of FIG. 2. In other words, the pitch motion means a motion of the end tool 100, which is formed to extend from the connection part 400, rotating vertically around the Y-axis with respect to the connection part 400.

Next, the yaw motion means a motion of the end tool 100 rotating in left and right directions, that is, a motion rotating around the Z-axis of FIG. 2, with respect to the extension direction of the connection part 400 (the X-axis direction of FIG. 2). In other words, the yaw motion means a motion of the end tool 100, which is formed to extend from the connection part 400, rotating horizontally around the Z-axis with respect to the connection part 400. That is, the yaw motion means rotating motions of two jaws 101 and 102, which are formed on the end tool 100, around the Z-axis in the same direction.

Meanwhile, the actuation motion means a motion of the end tool 100 rotating around the same shaft of rotation as that of the yaw motion, while the two jaws 101 and 102 rotating in the opposite directions so as to be closed or opened. That is, the actuation motion means rotating motions of the two jaws 101 and 102, which are formed on the end tool 100, in the opposite directions around the Z-axis.

In other words, the yaw rotation may be defined as a motion in which a jaw pulley to be described later rotates around a rotation shaft 141, which is a jaw pulley rotation shaft, and the pitch rotation may be defined as a motion in which the jaw pulley revolves around a rotation shaft 143, which is a pitch main rotation shaft.

The power transmission part 300 may serve to connect the manipulation part 200 to the end tool 100 to transmit the driving force of the manipulation part 200 to the end tool 100, and may include a plurality of wires, pulleys, links, sections, gears, and the like.

Meanwhile, for convenience of description, the plurality of wires, pulleys, and the like have been categorized as being included in the power transmission part 300, but the wires, pulleys, and the like on the end tool 100 side may be categorized as being included in the end tool 100, and those on the manipulation part 200 side may be categorized as being included in the manipulation part 200.

### Intuitive Driving

Hereinafter, intuitive driving of the surgical instrument 10 of the present disclosure will be described.

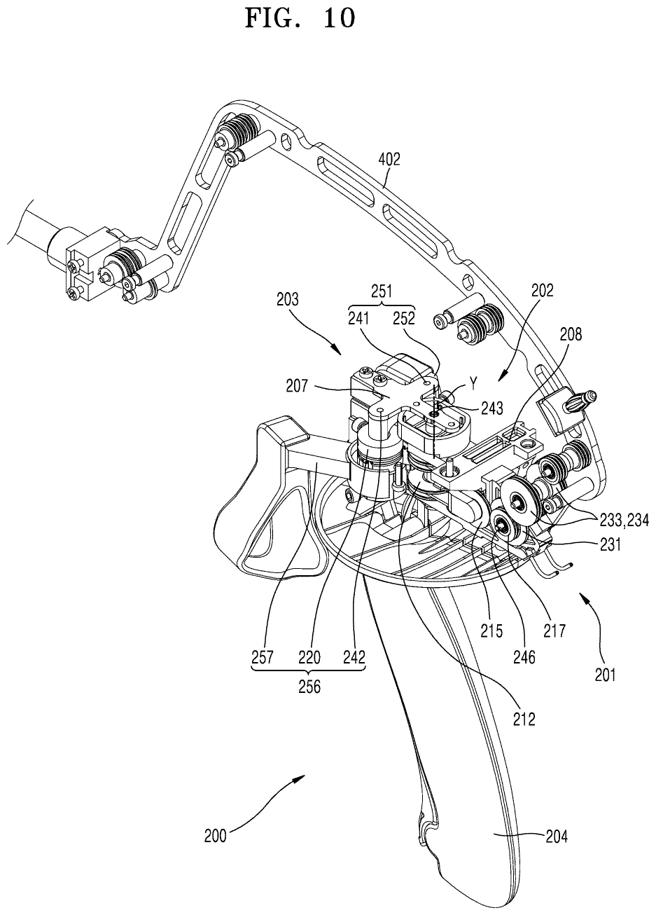

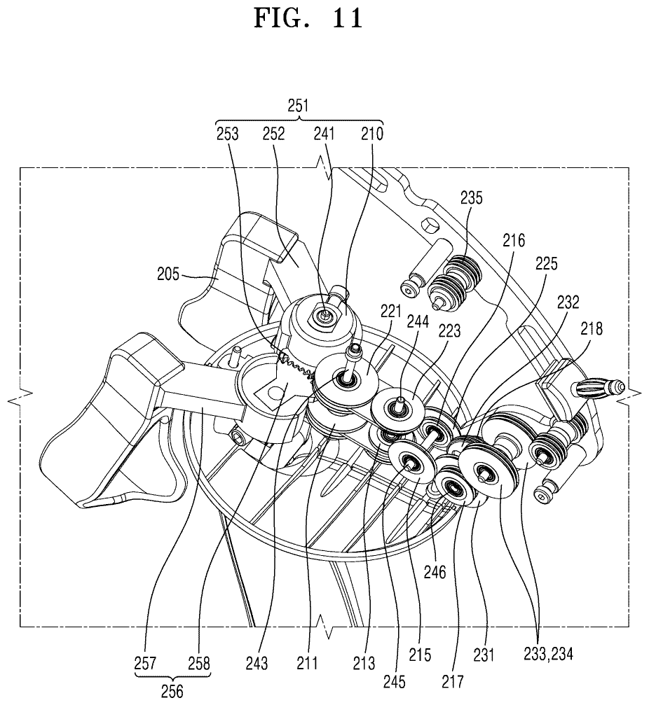

First, while holding a first handle 204 with the palm of the hand, the user may rotate the first handle 204 around the Y-axis (i.e., a rotation shaft 246) to perform a pitch motion, and rotate the first handle 204 around the Z-axis (i.e., a rotation shaft 243) to perform a yaw motion. In addition, referring to FIGS. 10 and 11 to be described later, the user may perform an actuation motion by manipulating the actuation manipulation part 203 while the thumb and index finger are inserted into a first actuation extension part 252 and/or a second actuation extension part 257, each of which is finger-ring-shaped and formed at one end portion of the actuation manipulation part 203.

Here, in the surgical instrument 10 according to an embodiment of the present disclosure, when the manipulation part 200 is rotated in one direction with respect to the connection part 400, the end tool 100 is rotated in a direction that is intuitively the same as a manipulation direction of the manipulation part 200. In other words, when the first handle 204 of the manipulation part 200 is rotated in one direction, the end tool 100 is also rotated in a direction intuitively the same as the one direction, so that a pitch motion or a yaw motion is performed. Here, the phrase “intuitively the same direction” may be further explained as meaning that a direction of movement of the user's finger gripping the manipulation part 200 and a direction of movement of a distal end of the end tool 100 form substantially the same direction. Of course, “the same direction” as used herein may not be a perfectly matching direction on a three-dimensional coordinate, and may be understood to be equivalent to the extent that, for example, when the user's finger moves to the left, the distal end of the end tool 100 is moved to the left, and when the user's finger moves down, the end portion of the end tool 100 is moved down.

In addition, to this end, in the surgical instrument 10 according to an embodiment of the present disclosure, the manipulation part 200 and the end tool 100 are formed in the same direction with respect to a plane perpendicular to the extension axis (an X-axis) of the connection part 400. That is, when viewed based on a YZ plane of FIG. 2, the manipulation part 200 is formed to extend in a positive (+) X-axis direction, and the end tool 100 is also formed to extend in the positive (+) X-axis direction. In other words, it may be said that a formation direction of the end tool 100 on one end portion of the connection part 400 is the same as a formation direction of the manipulation part 200 on another end portion of the connection part 400 on the basis of the YZ plane. Further, in other words, it may be said that the manipulation part 200 may be formed in a direction away from the body of a user holding the manipulation part 200, that is, in a direction in which the end tool 100 is formed. That is, in the parts such as the first handle 204, a first actuation manipulation part 251, a second actuation manipulation part 256, and the like, which are moved by the user's grip for actuation, yaw, and pitch motions, a corresponding portion that is moved for the motion is formed to extend in the positive (+) X-axis direction from the rotation center of a corresponding joint for the motion. In this manner, the manipulation part 200 may be configured in the same manner as the end tool 100 in which each moving portion is formed to extend in the positive (+) X-axis direction from the rotation center of a corresponding joint for the motion, and as described with reference to FIG. 1, the manipulation direction of the user may be identical to the operation direction of the end tool 100 from the viewpoint of the rotation directions and the left and right directions. As a result, intuitively the same manipulation may be achieved.

In detail, in the case of the conventional surgical instrument, a direction in which a user manipulates the manipulation part is different from a direction in which the end tool is actually operated, that is, intuitively different from the direction in which the end tool is actually operated, and thus, a surgical operator may not easily intuitively manipulate the surgical instrument and may spend a long time to learn a skill of operating the end tool in desired directions, and in some cases, malfunctions may occur, which may cause damage to patients.

In order to address such problems, the surgical instrument 10 according to an embodiment of the present disclosure is configured such that the manipulation direction of the manipulation part 200 and the operation direction of the end tool 100 are intuitively identical to each other. To this end, the manipulation part 200 is configured similar to the end tool 100, that is, in the manipulation part 200, parts that are actually moved for actuation, yaw, and pitch motions extend respectively from rotation centers of corresponding joints in the positive (+) X-axis direction.

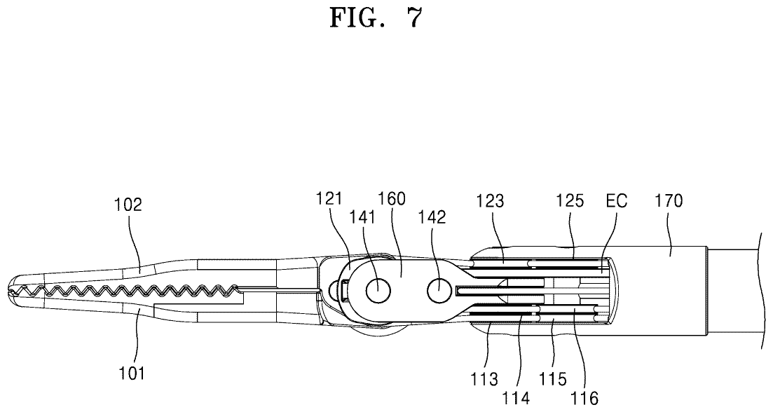

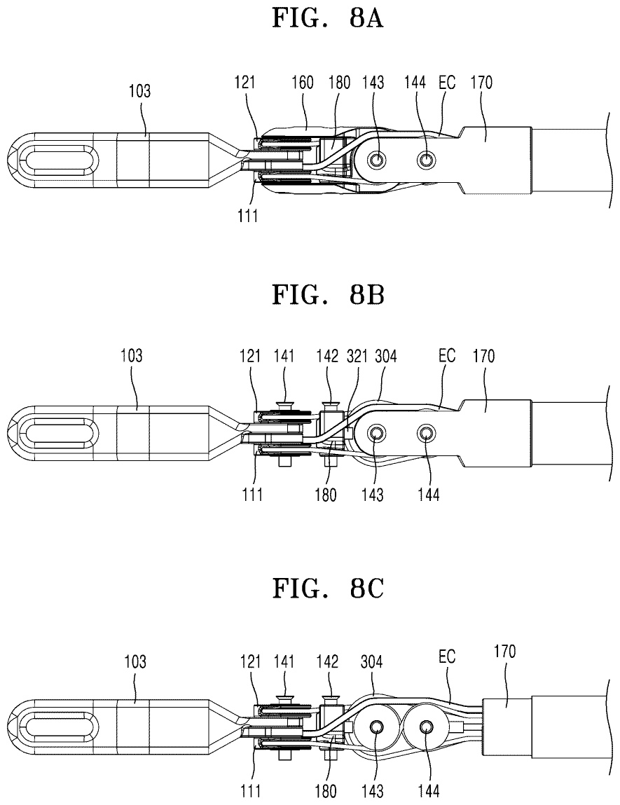

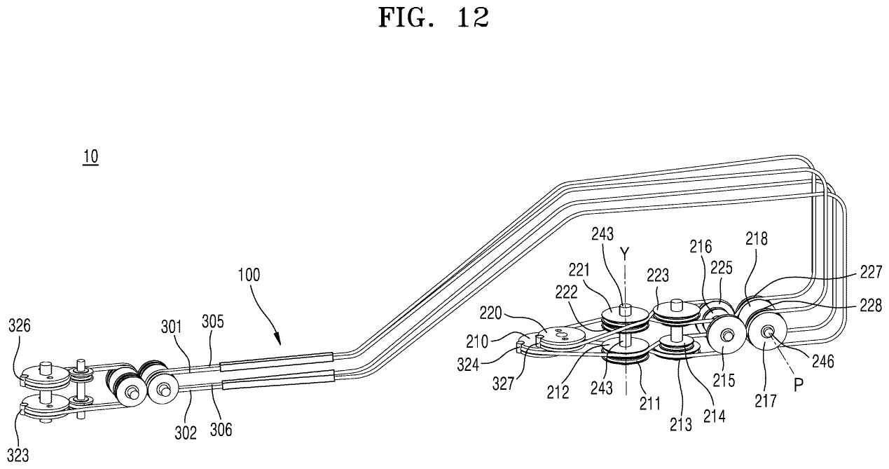

FIGS. 4 to 6 are perspective views illustrating the end tool 100 of the surgical instrument 10 according to an embodiment of the present disclosure. FIG. 7 is a plan view of the end tool 100 of the surgical instrument 10 of FIG. 4, and FIGS. 8A-8C are side views of the end tool 100 of the surgical instrument 10 of FIG. 4. FIGS. 9A and 9B are views illustrating an open state of the jaws 101 and 102 of the end tool 100 in the surgical instrument 10 shown in FIG. 7. FIGS. 10 and 11 are perspective views illustrating the manipulation part 200 of the surgical instrument 10 of FIG. 2, and FIG. 12 is a view schematically illustrating only a configuration of the pulleys and wires constituting the joints of the surgical instrument 10 illustrated in FIG. 2.

Hereinafter, the end tool 100, the manipulation part 200, and the power transmission part 300 of the surgical instrument 10 of FIG. 2 will be described in more detail with reference to FIGS. 4 to 12.

### Power Transmission Part

First, the power transmission part 300 of the surgical instrument 10 of FIG. 2 will be described in more detail.

The power transmission part 300 of the surgical instrument 10 according to an embodiment of the present disclosure may include a wire 301, a wire 302, a wire 303, a wire 304, a wire 305, and a wire 306.

Here, the wire 301 and the wire 305 may be paired to serve as first jaw wires. The wire 302 and the wire 306 may be paired to serve as second jaw wires. In this case, the components encompassing the wire 301 and the wire 305, which are first jaw wires, and the wire 302 and the wire 306, which are second jaw wires, may be referred to as jaw wires. In addition, the wire 303 and the wire 304 may be paired to serve as pitch wires.

In addition, the power transmission part 300 of the surgical instrument 10 according to an embodiment of the present disclosure may include a coupling member 321, a coupling member 323, a coupling member 324, a coupling member 326, and a coupling member 327 that are coupled to respective end portions of the wires to respectively couple the wires to the pulleys. Here, each of the coupling members may have various shapes as necessary, such as a ball shape, a tube shape, and the like.

Here, the coupling member 321 may serve as a pitch wire-end tool coupling member, the coupling member 323 may serve as a first jaw wire-end tool coupling member, and the coupling member 326 may serve as a second jaw wire-end tool coupling member.

In addition, the coupling member 324 serving as a first jaw wire-manipulation part coupling member, and the coupling member 327 serving as a second jaw wire-manipulation part coupling member are formed on the manipulation part 200 side, and although not shown in the drawings, and a pitch wire-manipulation part coupling member may be further formed on the manipulation part 200 side.

The coupling relationship between the wires, the coupling members, and the respectively pulleys will be described in detail as follows.

First, the wires 301 and 305, which are first jaw wires, may be a single wire. The coupling member 323, which is a first jaw wire-end tool coupling member, is inserted at an intermediate point of the first jaw wire, which is a single wire, and the coupling member 323 is crimped and fixed, and then, both strands of the first jaw wire centered on the coupling member 323 may be referred to as the wire 301 and the wire 305, respectively.

Alternatively, the wires 301 and 305, which are first jaw wires, may also be formed as separate wires and connected by the coupling member 323.

In addition, by coupling the coupling member 323 to a pulley 111, the wires 301 and 305 may be fixedly coupled to the pulley 111. This allows the pulley 111 to rotate as the wires 301 and 305 are pulled and released.

Meanwhile, the first jaw wire-manipulation part coupling member 324 may be coupled to another end portions of the wires 301 and 305, which are opposite to one end portions to which the coupling member 323 is coupled.

In addition, by coupling the first jaw wire-manipulation part coupling member 324 to a pulley 210, the wires 301 and 305 may be fixedly coupled to the pulley 210. As a result, when the pulley 210 is rotated by a motor or a human force, the pulley 111 of the end tool 100 may be rotated as the wire 301 and the wire 305 are pulled and released.

In the same manner, the wires 302 and 306, which are second jaw wires, are respectively coupled to the coupling member 326, which is a second jaw wire-end tool coupling member, and the second jaw wire-manipulation part coupling member 327. In addition, the coupling member 326 is coupled to a pulley 121, and the second jaw wire-manipulation part coupling member is coupled to a pulley 220. As a result, when the pulley 220 is rotated by a motor or a human force, the pulley 121 of the end tool 100 may be rotated as the wire 302 and the wire 306 are pulled and released.

In the same manner, the wires 303 and 304, which are pitch wires, are respectively coupled to the coupling member 321, which is a pitch wire-end tool coupling member, and the pitch wire-manipulation part coupling member (not shown). In addition, the coupling member 321 is coupled to a pulley 131, and the pitch wire-manipulation part coupling member (not shown) is coupled to a pulley 231. As a result, when the pulley 231 is rotated by a motor or a human force, the pulley 131 of the end tool 100 may be rotated as the wire 303 and the wire 304 are pulled and released.

As a result, the wire 301 and the wire 305, which are both strands of the first jaw wire, are coupled to the coupling member 323, which is a first jaw wire-end tool coupling member, and the coupling member (not shown), which is first jaw wire-driving part coupling member, so as to form as a whole a closed loop. Similarly, the second jaw wire and the pitch wire may each be configured to function as a closed loop.

### End Tool

The end tool 100 of the surgical instrument according to an embodiment of the present disclosure (hereinafter, referred to as an end tool) includes a pair of jaws for performing a grip motion, that is, a first jaw 101 and a second jaw 102. Here, each of the first jaw 101 and the second jaw 102, or a component encompassing the first jaw 101 and the second jaw 102 may be referred to as the jaw 103.

Further, the end tool 100 may include the pulley 111, a pulley 113, a pulley 114, a pulley 115, and a pulley 116 associated with a rotational motion of the first jaw 101. In addition, the end tool 100 may include the pulley 121, a pulley 123, a pulley 124, a pulley 125, and a pulley 126, which are associated with a rotational motion of the second jaw 102.

Here, the pulleys facing each other are illustrated in the drawings as being formed parallel to each other, but the concept of the present disclosure is not limited thereto, and each of the pulleys may be variously formed with a position and a size suitable for the configuration of the end tool.

Further, the end tool 100 of the present disclosure may include an end tool hub 160 and a pitch hub 170.

The rotation shaft 141 and a rotation shaft 142, which will be described later, may be inserted through the end tool hub 160, and the end tool hub 160 may internally accommodate at least some of the first jaw 101 and the second jaw 102, which are axially coupled to the rotation shaft 141.

In addition, the pulley 131 serving as an end tool pitch pulley may be formed at one end portion of the end tool hub 160. Here, the pulley 131 may be integrally formed with the end tool hub 160 as one body. That is, one end portion of the end tool hub 160 may be formed in a disk shape or a semi-circular shape, and a groove around which a wire may be wound may be formed on an outer circumferential surface of the end tool hub 160, thereby forming a kind of guide channel. Alternatively, the pulley 131 may be formed as a separate member from the end tool hub 160 to be coupled to the end tool hub 160. The wires 303 and 304 described above are coupled to the pulley 131 serving as an end tool pitch pulley, and a pitch motion is performed as the pulley 131 is rotated around the rotation shaft 143.

The rotation shaft 143 and a rotation shaft 144, which will be described later, may be inserted through the pitch hub 170, and the pitch hub 170 may be axially coupled to the end tool hub 160 and the pulley 131 by the rotation shaft 143. Thus, the end tool hub 160 and the pulley 131 (coupled thereto) may be configured to be rotatable around the rotation shaft 143 with respect to the pitch hub 170.

Further, the pitch hub 170 may internally accommodate at least some of the pulley 113, the pulley 114, the pulley 123, and the pulley 124 that are axially coupled to the rotation shaft 143. In addition, the pitch hub 170 may internally accommodate at least some of the pulley 115, the pulley 116, the pulley 125, and the pulley 126 that are axially coupled to the rotation shaft 144.

Further, the end tool 100 of the present disclosure may include the rotation shaft 141, the rotation shaft 142, the rotation shaft 143, and the rotation shaft 144. As described above, the rotation shaft 141 and the rotation shaft 142 may be inserted through the end tool hub 160, and the rotation shaft 143 and the rotation shaft 144 may be inserted through the pitch hub 170.

The rotation shaft 141, the rotation shaft 142, the rotation shaft 143, and the rotation shaft 144 may be arranged sequentially from a distal end 104 toward a proximal end 105 of the end tool 100. Accordingly, starting from the distal end 104, the rotation shaft 141 may be referred to as a first pin, the rotation shaft 142 may be referred to as a second pin, the rotation shaft 143 may be referred to as a third pin, and the rotation shaft 144 may be referred to as a fourth pin.

Here, the rotation shaft 141 may function as an end tool jaw pulley rotation shaft, the rotation shaft 142 may function as an end tool jaw auxiliary pulley rotation shaft, the rotation shaft 143 may function as an end tool pitch rotation shaft, and the rotation shaft 144 may function as an end tool pitch auxiliary rotation shaft of the end tool 100.

Each of the rotation shafts 141, 142, 143, and 144 may be fitted into one or more pulleys, which will be described in detail below.

The pulley 111 functions as an end tool first jaw pulley, and the pulley 121 functions as an end tool second jaw pulley, and these two components may be collectively referred to as end tool jaw pulleys.

The pulley 111 and the pulley 121, which are end tool jaw pulleys, are configured to face each other, and are configured to be rotatable independently of each other around the rotation shaft 141, which is an end tool jaw pulley rotation shaft.

In the drawings, it is illustrated that the pulley 111 and the pulley 121 are configured to rotate around one rotation shaft 141, but it is of course possible that each jaw pulley may be configured to be rotatable around a separate shaft. Here, the first jaw 101 may be fixedly coupled to the pulley 111 and rotated together with the pulley 111, and the second jaw 102 may be fixedly coupled to the pulley 121 and rotated together with the pulley 121. Yaw and actuation motions of the end tool 100 are performed in response to the rotation the pulley 111 and the pulley 121. That is, when the pulley 111 and the pulley 121 are rotated in the same direction around the rotation shaft 141, the yaw motion is performed, and when the pulley 111 and the pulley 121 are rotated in opposite directions around the rotation shaft 141, the actuation motion is performed.

Here, the first jaw 101 and the pulley 111 may be formed as separate members and coupled to each other, or the first jaw 101 and the pulley 111 may be integrally formed as one body. Similarly, the second jaw 102 and the pulley 121 may be formed as separate members and coupled to each other, or the second jaw 102 and the pulley 121 may be integrally formed as one body.

The pulley 113 and the pulley 114 function as end tool first jaw pitch main pulleys, and the pulley 123 and the pulley 124 function as end tool second jaw pitch main pulleys, and these two components may be referred to collectively as an end tool jaw pitch main pulley. At this time, the rotation shaft 143 may be disposed to pass through the pulley 113, the pulley 114, the pulley 123, and the pulley 124.

The pulley 115 and the pulley 116 function as end tool first jaw pitch sub-pulleys, and the pulley 125 and the pulley 126 function as end tool second jaw pitch sub-pulleys, and these two components may be collectively referred to as an end tool jaw pitch sub-pulley. At this time, the rotation shaft 144 may be disposed to pass through the pulley 115, the pulley 116, the pulley 125, and the pulley 126.

Further, the pulley 113, the pulley 114, the pulley 123, the pulley 124, the pulley 115, the pulley 116, the pulley 125, and the pulley 126 may be collectively referred to as end tool jaw pitch pulleys.

As a result, the rotation shaft 141, the rotation shaft 142, the rotation shaft 143, and the rotation shaft 144 may be arranged sequentially from the distal end 104 of the end tool 100 toward the proximal end 105.

In addition, the pulley 111, the pulley 113/pulley 114, and the pulley 115/pulley 116, which are pulleys associated with the rotation of the first jaw 101, may be arranged sequentially from the distal end 104 toward the proximal end 105 of the end tool 100.

In addition, the pulley 121, the pulley 123/pulley 124, and the pulley 125/pulley 126, which are pulleys associated with the rotation of the second jaw 102, may be arranged sequentially from the distal end 104 toward the proximal end 105 of the end tool 100.

Hereinafter, components related to the rotation of the pulley 111 will be described.

The pulley 113 and the pulley 114 function as end tool first jaw pitch main pulleys. That is, the pulley 113 and the pulley 114 function as main rotation pulleys for a pitch motion of the first jaw 101. Here, the wire 301, which is a first jaw wire, is wound around the pulley 113, and the wire 305, which is a first jaw wire, is wound around the pulley 114.

The pulley 115 and the pulley 116 function as end tool first jaw pitch sub-pulleys. That is, the pulley 115 functions as a sub rotation pulley for a pitch motion of the first jaw 101. Here, the wire 301, which is a first jaw wire, is wound around the pulley 115, and the wire 305, which is a first jaw wire, is wound around the pulley 116.

Here, the pulley 113 and the pulley 114 are disposed on one side of the pulley 111 and a pulley 112 to face each other. Here, the pulley 113 and the pulley 114 are configured to be rotatable independently of each other around the rotation shaft 143 that is an end tool pitch rotation shaft. In addition, the pulley 115 and the pulley 116 are disposed on one side of the pulley 113 and one side of the pulley 114, respectively, to face each other. Here, the pulley 115 and the pulley 116 are configured to be rotatable independently of each other around the rotation shaft 144 that is an end tool pitch auxiliary rotation shaft. Here, in the drawings, it is illustrated that the pulley 113, the pulley 115, the pulley 114, and the pulley 116 are all configured to be rotatable around the Y-axis direction, but the concept of the present disclosure is not limited thereto, and the rotation shafts of the respective pulleys may be formed in various directions according to configurations thereof.

The wire 301, which is a first jaw wire, is sequentially wound to make contact with at least portions of the pulley 115, the pulley 113, and the pulley 111. In addition, the wire 305 connected to the wire 301 by the coupling member 323 is sequentially wound to make contact with at least portions of the pulley 111, the pulley 112, the pulley 114, and the pulley 116.

In other words, the wires 301 and 305, which are first jaw wires, are sequentially wound to make contact with at least portions of the pulley 115, the pulley 113, the pulley 111, the pulley 112, the pulley 114, and the pulley 116 and are configured to move along the above pulleys while rotating the above pulleys.

Accordingly, when the wire 301 is pulled in the direction of an arrow 301 of FIGS. 9A and 9B, a coupling member 323 to which the wire 301 is coupled and the pulley 111 coupled to the coupling member 323 are rotated in an arrow L direction of FIGS. 9A and 9B. In contrast, when the wire 305 is pulled in the direction of an arrow 305 of FIGS. 9A and 9B, the coupling member 323 to which the wire 305 is coupled and the pulley 111 coupled to the coupling member 323 are rotated in an arrow R direction of FIGS. 9A and 9B.

Next, components related to the rotation of the pulley 121 will be described.

The pulley 123 and the pulley 124 function as end tool second jaw pitch main pulleys. That is, the second jaw 102 functions as a main rotation pulley for a pitch motion of the second jaw 102. Here, the wire 306, which is a second jaw wire, is wound around the pulley 123, and the wire 302, which is a second jaw wire, is wound around the pulley 124.

The pulley 125 and the pulley 126 function as end tool second jaw pitch sub-pulleys. That is, the pulley 125 and the pulley 126 function as sub rotation pulleys for a pitch motion of the second jaw 102. Here, the wire 306, which is a second jaw wire, is wound around the pulley 125, and the wire 302, which is a second jaw wire, is wound around the pulley 126.

Here, the pulley 123 and the pulley 124 are disposed on one side of the pulley 121 and a pulley 122 to face each other. Here, the pulley 123 and the pulley 124 are configured to be rotatable independently of each other around the rotation shaft 143 which is an end tool pitch rotation shaft. In addition, the pulley 125 and the pulley 126 are disposed on one side of the pulley 123 and one side of the pulley 124, respectively, to face each other. Here, the pulley 125 and the pulley 126 are configured to be rotatable independently of each other around the rotation shaft 144 that is an end tool pitch auxiliary rotation shaft. Here, in the drawings, it is illustrated that all of the pulley 123, the pulley 125, the pulley 124, and the pulley 126 are configured to be rotatable around the Y-axis direction, but the concept of the present disclosure is not limited thereto, and the rotating axes of the respective pulleys may be formed in various directions according to configurations thereof.

The wire 306, which is a second jaw wire, is sequentially wound to make contact with at least portions of the pulley 125, the pulley 123, and the pulley 121. In addition, the wire 302 connected to the wire 306 by the coupling member 326 is sequentially wound to make contact with at least portions of the pulley 121, the pulley 122, the pulley 124, and the pulley 126.

In other words, the wires 306 and 302, which are second jaw wires, are sequentially wound to make contact with at least portions of the pulley 125, the pulley 123, the pulley 121, the pulley 122, the pulley 124, and the pulley 126, and are configured to move along the above pulleys while rotating the above pulleys.

Accordingly, when the wire 306 is pulled in the direction of an arrow 306 of FIGS. 9A and 9B, the coupling member 326 to which the wire 306 is coupled and the pulley 121 coupled to the coupling member 326 are rotated in the arrow R direction of FIGS. 9A and 9B. In contrast, when the wire 302 is pulled in the direction of an arrow 302 of FIGS. 9A and 9B, the coupling member 326 to which the wire 302 is coupled and the pulley 121 coupled to the coupling member 326 are rotated in the arrow L direction of FIGS. 9A and 9B.

Hereinafter, a pitch motion of the present disclosure will be described in more detail.

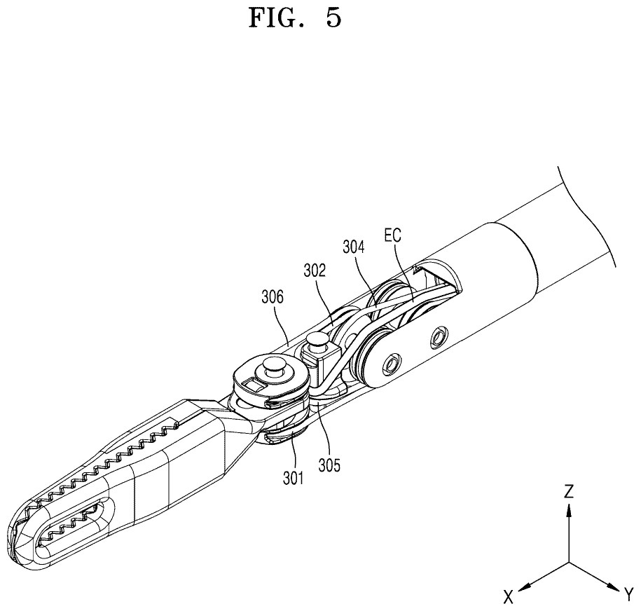

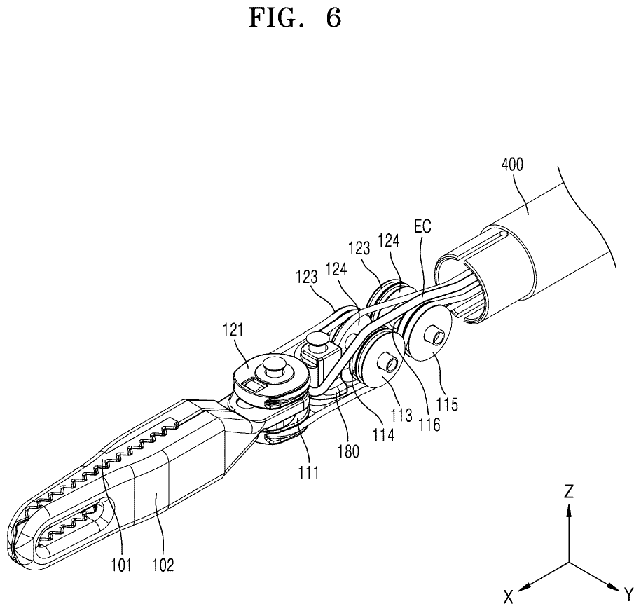

When the wire 301 is pulled toward the arrow 301 of FIGS. 9A and 9B, and simultaneously, the wire 305 is pulled toward the arrow 305 of FIGS. 9A and 9B (that is, when both strands of the first jaw wire are pulled), as shown in FIGS. 5 and 6, since the wires 301 and 305 are wound around lower portions of the pulley 113 and the pulley 114 rotatable around the rotation shaft 143, which is an end tool pitch rotation shaft, the pulley 111 to which the wires 301 and 305 are fixedly coupled and the end tool hub 160 to which the pulley 111 is coupled are rotated as a whole in the counterclockwise direction around the rotation shaft 143, so that the end tool 100 is rotated downward to perform a pitch motion. At this time, since the second jaw 102 and the wires 302 and 306 fixedly coupled thereto are wound around upper portions of the pulley 123 and the pulley 124 rotatable around the rotation shaft 143, the wires 302 and 306 are unwound in an opposite directions of the arrows 302 and 306, respectively.

In contrast, when the wire 302 is pulled toward the arrow 302 of FIGS. 9A and 9B, and simultaneously, the wire 306 is pulled toward the arrow 306 of FIGS. 9A and 9B, as shown in FIGS. 5 and 6, since the wires 302 and 306 are wound around the upper portions of the pulley 123 and the pulley 124 rotatable around the rotation shaft 143, which is an end tool pitch rotation shaft, the pulley 121 to which the wires 302 and 306 are fixedly coupled and the end tool hub 160 to which the pulley 121 is coupled are rotated as a whole in the clockwise direction around the rotation shaft 143, so that the end tool 100 is rotated upward to perform a pitch motion. At this time, since the first jaw 101 and the wires 301 and 305 fixedly coupled thereto are wound around lower portions of the pulley 113 and the pulley 114 rotatable around the rotation shaft 143, the wires 302 and 306 are moved in opposite directions of the wires 301 and 305, respectively.

Meanwhile, the end tool 100 of the surgical instrument 10 of the present disclosure may further include the pulley 131, which is an end tool pitch pulley, the manipulation part 200 may further include the pulley 231 and a pulley 232, which are manipulation part pitch pulleys, and the power transmission part 300 may further include the wires 303 and 304, which are pitch wires. In detail, the pulley 131 of the end tool 100 is rotatable around the rotation shaft 143, which is an end tool pitch rotation shaft, and may be integrally formed with the end tool hub 160 (or fixedly coupled to the end tool hub 160) as one body. In addition, the wires 303 and 304 may serve to connect the pulley 131 of the end tool 100 to the pulley 231 and the pulley 232 of the manipulation part 200.

Thus, when the pulley 231 and the pulley 232 of the manipulation part 200 are rotated, the rotation of the pulley 231 and the pulley 232 is transmitted to the pulley 131 of the end tool 100 through the wires 303 and 304 so that the pulley 131 is rotated together therewith, and as a result, the end tool 100 performs a pitch motion while rotating.

That is, in the surgical instrument 10 according to an embodiment of the present disclosure, by providing the pulley 131 of the end tool 100, the pulley 231, and the pulley 232 of the manipulation part 200, and the wires 303 and 304 of the power transmission part 300 to transmit power for a pitch motion, the driving force for the pitch motion of the manipulation part 200 may be more completely transmitted to the end tool 100, thereby improving operation reliability.

Here, a diameter of each of the pulley 113, the pulley 114, the pulley 123, and the pulley 124, which are end tool jaw pitch main pulleys, and a diameter of the pulley 131, which is an end tool pitch pulley, may be the same as each other or different from each other. At this time, a ratio of the diameter of the end tool jaw pitch main pulley to the diameter of the end tool pitch pulley may be the same as a ratio of a diameter of the manipulation part pitch pulley of the manipulation part 200 to a diameter of a manipulation part pitch main pulley to be described later.

### Manipulation Part

The manipulation part 200 of the surgical instrument 10 according to an embodiment of the present disclosure includes the first handle 204 that can be gripped by a user, an actuation manipulation part 203 that controls an actuation motion of the end tool 100, the yaw manipulation part 202 that controls a yaw motion of the end tool 100, and a pitch manipulation part 201 that controls a pitch motion of the end tool 100.

The manipulation part 200 may include the pulley 210, a pulley 211, a pulley 212, a pulley 213, a pulley 214, a pulley 215, a pulley 216, a pulley 217, and a pulley 218 that are related to a rotational motion of the first jaw 101. In addition, the manipulation part 200 may include the pulley 220, a pulley 221, a pulley 222, a pulley 223, a pulley 224, a pulley 225, a pulley 226, a pulley 227, and a pulley 228 that are related to a rotational motion of the second jaw 102. In addition, the manipulation part 200 may include the pulley 213, the pulley 232, a pulley 233, and a pulley 234 that are related to a pitch motion of the second jaw 102. In addition, the manipulation part 200 may include a pulley 235, which is a relay pulley disposed at some places along the bent part 402 of the connection part 400.

Here, the pulleys facing each other are illustrated in the drawings as being formed parallel to each other, but the concept of the present disclosure is not limited thereto, and each of the pulleys may be variously formed with a position and a size suitable for the configuration of the manipulation part. Further, the manipulation part 200 of the first embodiment of the present disclosure may include a rotation shaft 241, a rotation shaft 242, the rotation shaft 243, a rotation shaft 244, a rotation shaft 245, and the rotation shaft 246. Here, the rotation shaft 241 may function as a manipulation part first jaw actuation rotation shaft, and the rotation shaft 242 may function as a manipulation part second jaw actuation rotation shaft. In addition, the rotation shaft 243 may function as a manipulation part yaw main rotation shaft, and the rotation shaft 244 may function as a manipulation part yaw sub-rotation shaft. In addition, the rotation shaft 245 may function as a manipulation part pitch sub-rotation shaft, and the rotation shaft 246 may function as a manipulation part pitch main rotation shaft.

The rotation shaft 241, the rotation shaft 242, the rotation shaft 243, the rotation shaft 244, the rotation shaft 245, and the rotation shaft 246 may be sequentially disposed from a distal end 205 toward a proximal end 206 of the manipulation part 200.

Each of the rotation shafts 241, 242, 243, 244, 245, and 246 may be fitted into one or more pulleys, which will be described in detail later.

The pulley 210 functions as a manipulation part first jaw actuation pulley, the pulley 220 functions as a manipulation part second jaw actuation pulley, and these components may also be collectively referred to as a manipulation part actuation pulley.

The pulley 211 and the pulley 212 function as manipulation part first jaw yaw main pulleys, the pulley 221 and the pulley 222 function as manipulation part second jaw yaw main pulleys, and these components may also be collectively referred to as a manipulation part yaw main pulley.

The pulley 213 and the pulley 214 function as manipulation part first jaw yaw sub-pulleys, the pulley 223 and the pulley 224 function as manipulation part second jaw yaw sub-pulleys, and these components may also be collectively referred to as a manipulation part yaw sub-pulley.

The pulley 215 and the pulley 216 function as manipulation part first jaw pitch sub-pulleys, the pulley 225 and the pulley 226 function as manipulation part second jaw pitch sub-pulleys, and these components may also be collectively referred to as a manipulation part pitch sub-pulley.

The pulley 217 and the pulley 218 function as manipulation part first jaw pitch main pulleys, and the pulley 227 and the pulley 228 function as manipulation part second jaw pitch main pulleys, and these components may also be collectively referred to as a manipulation part pitch main pulley.

The pulley 231 and the pulley 232 function as manipulation part pitch main pulleys, and the pulley 233 and the pulley 234 function as manipulation part pitch sub-pulleys.

The above components are categorized from the perspective of the manipulation part for each motion (pitch/yaw/actuation) as follows.

The pitch manipulation part 201 configured to control a pitch motion of the end tool 100 may include the pulley 215, the pulley 216, the pulley 217, the pulley 218, the pulley 225, the pulley 226, the pulley 227, the pulley 228, the pulley 231, the pulley 232, the pulley 233, and the pulley 234. In addition, the pitch manipulation part 201 may include the rotation shaft 245 and the rotation shaft 246. In addition, the pitch manipulation part 201 may further include a pitch frame 208.

The yaw manipulation part 202 configured to control a yaw motion of the end tool 100 may include the pulley 211, the pulley 212, the pulley 213, the pulley 214, the pulley 221, the pulley 222, the pulley 223, and the pulley 224. In addition, the yaw manipulation part 202 may include the rotation shaft 243 and the rotation shaft 244. In addition, the yaw manipulation part 202 may further include a yaw frame 207.

The actuation manipulation part 203 configured to control an actuation motion of the end tool 100 may include the pulley 210, the pulley 220, the rotation shaft 241, and the rotation shaft 242. In addition, the actuation manipulation part 203 may further include the first actuation manipulation part 251 and the second actuation manipulation part 256.

Hereinafter, each component of the manipulation part 200 will be described in more detail.

The first handle 204 may be configured to be gripped by a user with the hand, and in particular, may be configured to be grasped by the user by wrapping the first handle 204 with his/her palm. In addition, the actuation manipulation part 203 and the yaw manipulation part 202 are formed on the first handle 204, and the pitch manipulation part 201 is formed on one side of the yaw manipulation part 202. In addition, another end portion of the pitch manipulation part 201 is connected to the bent part 402 of the connection part 400.

The actuation manipulation part 203 includes the first actuation manipulation part 251 and the second actuation manipulation part 256. The first actuation manipulation part 251 includes the rotation shaft 241, the pulley 210, the first actuation extension part 252, and a first actuation gear 253. The second actuation manipulation part 256 includes the rotation shaft 242, the pulley 220, the second actuation extension part 257, and a second actuation gear 258. Here, end portions of the first actuation extension part 252 and the second actuation extension part 257 are each formed in the shape of a hand ring, and may act as a second handle.

Here, the rotation shaft 241 and the rotation shaft 242, which are actuation rotation axes, may be configured to form a predetermined angle with an XY plane on which the connection part 400 is formed. For example, the rotation shaft 241 and the rotation shaft 242 may be formed in a direction parallel to the Z-axis, and in this state, when the pitch manipulation part 201 or the yaw manipulation part 202 is rotated, a coordinate system of the actuation manipulation part 203 may change relatively. Of course, the concept of the present disclosure is not limited thereto, and the rotation shaft 241 and the rotation shaft 242 may be formed in various directions so as to be suitable for a structure of the hand of the user gripping the actuation manipulation part 203 according to an ergonomic design.

Meanwhile, the pulley 210, the first actuation extension part 252, and the first actuation gear 253 are fixedly coupled to each other to be rotatable together around the rotation shaft 241. Here, the pulley 210 may be configured as a single pulley or two pulleys fixedly coupled to each

Similarly, the pulley 220, a second actuation extension part 257, and a second actuation gear 258 are fixedly coupled to each other to be rotatable together around the rotation shaft 242. Here, the pulley 220 may be configured as a single pulley or two pulleys fixedly coupled to each other.

Here, the first actuation gear 253 and the second actuation gear 258 are configured to be engaged with each other such that, when any one gear is rotated in one direction, another gear is rotated together with the one gear in a direction opposite to the one direction.

The yaw manipulation part 202 may include the rotation shaft 243, the pulleys 211 and 212, which are manipulation part first jaw yaw main pulleys, the pulleys 221 and 222, which are manipulation part second jaw yaw main pulleys, and the yaw frame 207. In addition, the yaw manipulation part 202 may further include the pulleys 213 and 214, which are manipulation part first jaw yaw sub-pulleys formed on one side of the pulley 211 and one side of the pulley 212, respectively, and the pulleys 223 and 224 that are manipulation part second jaw yaw sub-pulleys formed on one side of the pulley 221 and one side of the pulley 222, respectively. Here, the pulleys 213 and 214 and the pulleys 223 and 224 may be coupled to the pitch frame 208 to be described later.

Here, it is illustrated in the drawings that the yaw manipulation part 202 includes the pulleys 211 and 212 and the pulleys 221 and 222, wherein the pulleys 211 and 212 and the pulleys 221 and 222 are each provided with two pulleys configured to face each other and independently rotatable, but the concept of the present disclosure is not limited thereto. That is, one or more pulleys having the same diameter or different diameters may be provided according to the configuration of the yaw manipulation part 202.

In detail, the rotation shaft 243, which is a manipulation part yaw main rotation shaft, is formed on one side of the actuation manipulation part 203 on the first handle 204. At this time, the first handle 204 is configured to be rotatable around the rotation shaft 243.

Here, the rotation shaft 243 may be configured to form a predetermined angle with the XY plane on which the connection part 400 is formed. For example, the rotation shaft 243 may be formed in a direction parallel to the Z-axis, and in this state, when the pitch manipulation part 201 is rotated, a coordinate system of the rotation shaft 243 may change relatively as described above. Of course, the concept of the present disclosure is not limited thereto, and the rotation shaft 243 may be formed in various directions so as to be suitable for a structure of the hand of the user gripping the manipulation part 200 according to an ergonomic design.

Meanwhile, the pulleys 211 and 212 and the pulleys 221 and 222 are coupled to the rotation shaft 243 so as to be rotatable around the rotation shaft 243. In addition, the wire 301 or the wire 305, which is a first jaw wire, is wound around the pulleys 211 and 212, and the wire 302 or the wire 306, which is a second jaw wire, may be wound around the pulleys 221 and 222. In this case, the pulleys 211 and 212 and the pulleys 221 and 222 may each be configured as two pulleys configured to face each other and independently rotatable. Accordingly, a wire being wound and a wire being released may be wound around respective separate pulleys so that the wires may perform motions without interference with each other.

The yaw frame 207 rigidly connects the first handle 204, the rotation shaft 241, the rotation shaft 242, and the rotation shaft 243 to each other, so that the first handle 204, the yaw manipulation part 202, and the actuation manipulation part 203 are integrally yaw-rotated around the rotation shaft 243.

The pitch manipulation part 201 may include the rotation shaft 246, the pulley 217 and the pulley 218, which are manipulation part first jaw pitch main pulleys, the pulleys 227 and 228, which are manipulation part second jaw pitch main pulleys, and the pitch frame 208. In addition, the pitch manipulation part 201 may further include the rotation shaft 245, the pulleys 215 and 216, which are manipulation part first jaw pitch sub-pulleys formed on one side of the pulley 217 and one side of the pulley 218, respectively, and the pulleys 225 and 226, which are manipulation part second jaw pitch sub-pulleys formed on one side of the pulley 227 and one side of the pulley 228, respectively. The pitch manipulation part 201 may be connected to the bent part 402 of the connection part 400 through the rotation shaft 246.

In detail, the pitch frame 208 is a base frame of the pitch manipulation part 201, and the rotation shaft 243 is rotatably coupled to one end portion thereof. That is, the yaw frame 207 is configured to be rotatable around the rotation shaft 243 with respect to the pitch frame 208.

As described above, since the yaw frame 207 connects the first handle 204, the rotation shaft 243, the rotation shaft 241, and the rotation shaft 242 to each other, and the yaw frame 207 is also axially coupled to the pitch frame 208, when the pitch frame 208 is pitch-rotated around the rotation shaft 246, the yaw frame 207 connected to the pitch frame 208, the first handle 204, the rotation shaft 241, the rotation shaft 242, and the rotation shaft 243 are pitch-rotated together with the pitch frame 208. That is, when the pitch manipulation part 201 is rotated around the rotation shaft 246, the actuation manipulation part 203 and the yaw manipulation part 202 are rotated together with the pitch manipulation part 201. In other words, when a user pitch-rotates the first handle 204 around the rotation shaft 246, the actuation manipulation part 203, the yaw manipulation part 202, and the pitch manipulation part 201 are moved together with the first handle 204.

The pulleys 217 and 218 and the pulleys 227 and 228 are coupled to the rotation shaft 246 so as to be rotatable around the rotation shaft 246 of the pitch frame 208.

Here, the pulley 217 and the pulley 218 may be configured to face each other so as to be independently rotatable. Accordingly, a wire being wound and a wire being released may be wound around respective separate pulleys so that the wires may perform motions without interference with each other. Similarly, the pulley 227 and the pulley 228 may also be configured to face each other so as to be independently rotatable. Accordingly, a wire being wound and a wire being released may be wound around respective separate pulleys so that the wires may perform motions without interference with each other.

A motion of each of the wires 303 and 304, which are pitch wires, is described as follows.

The pulley 131, which is an end tool pitch pulley, is fixedly coupled to the end tool hub 160 in the end tool 100, and the pulley 231 and the pulley 232, which are manipulation part pitch pulleys, are fixedly coupled to the pitch frame 208 in the manipulation part 200. In addition, these pulleys are connected to each other by the wires 303 and 304, which are pitch wires, so that a pitch motion of the end tool 100 may be performed more easily according to the pitch manipulation of the manipulation part 200. Here, the wire 303 is fixedly coupled to the pitch frame 208 via the pulley 231 and the pulley 233, and the wire 304 is fixedly coupled to the pitch frame 208 via the pulley 232 and the pulley 234. That is, the pitch frame 208 and the pulleys 231 and 232 are rotated together around the rotation shaft 246 by the pitch rotation of the manipulation part 200, and as a result, the wires 303 and 304 are also moved, and thus, a driving force of additional pitch rotation may be transmitted separately from the pitch motion of the end tool 100 by the wire 301, the wire 302, the wire 305, and the wire 306, which are jaw wires.

A connection relationship of each of the first handle 204, the pitch manipulation part 201, the yaw manipulation part 202, and the actuation manipulation part 203 is summarized as follows. The rotation shafts 241 and 242, the rotation shaft 243, the rotation shaft 244, the rotation shaft 245, and the rotation shaft 246 may be formed on the first handle 204. In this case, since the rotation shafts 241 and 242 are directly formed on the first handle 204, the first handle 204 and the actuation manipulation part 203 may be directly connected to each other. In addition, since the rotation shaft 243 is directly formed on the first handle 204, the first handle 204 and the yaw manipulation part 202 may be directly connected to each other. On the other hand, since the pitch manipulation part 201 is formed on one side of the yaw manipulation part 202 so as to be connected to the yaw manipulation part 202, the pitch manipulation part 201 is not directly connected to the first handle 204, and the pitch manipulation part 201 and the first handle 204 may be configured to be indirectly connected to each other via the yaw manipulation part 202.

Continuing to refer to the drawings, in the surgical instrument 10 according to an embodiment of the present disclosure, the pitch manipulation part 201 and the end tool 100 may be formed on the same or parallel axis (the X-axis). That is, the rotation shaft 246 of the pitch manipulation part 201 is formed at one end portion of the bent part 402 of the connection part 400, and the end tool 100 is formed at another end portion of the connection part 400.

In addition, one or more relay pulleys 235 configured to change or guide paths of the wires may be disposed at some places along the connection part 400, particularly in the bent part 402. By positioning at least some of the wires to wound around the relay pulleys 235 to guide the paths of the wires, these wires may be placed along a bent shape of the bent part 402.

Here, in the drawings, it is illustrated that the connection part 400 is formed to be curved with a predetermined curvature by having the bent part 402, but the concept of the present disclosure is not limited thereto, and the connection part 400 may be formed linearly or to be bent one or more times as necessary, and even in this case, it may be said that the pitch manipulation part 201 and the end tool 100 are formed on substantially the same axis or parallel axes. In addition, although FIG. 5 illustrates that each of the pitch manipulation part 201 and the end tool 100 is formed on an axis parallel to the X-axis, the concept of the present disclosure is not limited thereto, and the pitch manipulation part 201 and the end tool 100 may be formed on different axes.

### Actuation, Yaw, and Pitch Motions

Actuation, yaw, and pitch motions in the present embodiment will be described as follows.

First, the actuation motion is described as follows.

In a state in which a user inserts his/her index finger in the hand ring formed on the first actuation extension part 252 and his/her thumb in the hand ring formed on the second actuation extension part 257, when the user rotates the actuation extension parts 252 and 257 using one or both of his/her index finger and thumb, the pulley 210 and the first actuation gear 253 fixedly coupled to the first actuation extension part 252 are rotated around the rotation shaft 241, and the pulley 220 and the second actuation gear 258 fixedly coupled to the second actuation extension part 257 are rotated around the rotation shaft 242. At this time, the pulley 210 and the pulley 220 are rotated in opposite directions, and thus the wires 301 and 305 fixedly coupled to the pulley 210 at one end portion thereof and the wires 302 and 306 fixedly coupled to the pulley 220 at one end portion thereof are also moved in opposite directions. In addition, a rotating force is transmitted to the end tool 100 through the power transmission part 300, and two jaws 101 and 102 of the end tool 100 perform the actuation motion.

Here, as described above, the actuation motion refers to a motion in which the two jaws 101 and 102 are splayed or closed while being rotated in opposite directions. That is, when the actuation extension parts 252 and 257 of the actuation manipulation part 203 are rotated in directions close to each other, the first jaw 101 is rotated in the counterclockwise direction, and the second jaw 102 is rotated in the clockwise direction, thereby closing the end tool 100. On the contrary, when the actuation extension parts 252 and 257 of the actuation manipulation part 203 are rotated in directions away from each other, the first jaw 101 is rotated in the counterclockwise direction, and the second jaw 102 is rotated in the clockwise direction, thereby opening the end tool 100.

In the present embodiment, for the actuation manipulation described above, the first actuation extension part 252 and the second actuation extension part 257 are provided to configure the second handle and manipulated by gripping the second handle with two fingers. However, for the actuation manipulation in which the two jaws of the end tool 100 are opened or closed, the actuation manipulation part 203 may be configured in a manner different from the above-described manner, such as configuring the two actuation pulleys (the pulley 210 and the pulley 220) to act in opposition to each other with one actuation rotation part.

Next, the yaw motion is described as follows.

When a user rotates the first handle 204 around the rotation shaft 243 while holding the first handle 204, the actuation manipulation part 203 and the yaw manipulation part 202 are yaw-rotated around the rotation shaft 243. That is, when the pulley 210 of the first actuation manipulation part 251 to which the wires 301 and 305 are fixedly coupled is rotated around the rotation shaft 243, the wires 301 and 305 wound around the pulleys 211 and 212 are moved. Similarly, when the pulley 220 of the second actuation manipulation part 256, to which the wires 302 and 306 are fixedly coupled, is rotated around the rotation shaft 243, the wires 302 and 306 wound around the pulleys 221 and 222 are moved. At this time, the wires 301 and 305 connected to the first jaw 101 and the wires 302 and 306 connected to the second jaw 102 are wound around the pulleys 211 and 212 and the pulleys 221 and 222, so that the first jaw 101 and the second jaw 102 are rotated in the same direction during the yaw rotation. In addition, a rotating force is transmitted to the end tool 100 through the power transmission part 300, and thus a yaw motion in which two jaws 103 of the end tool 100 are rotated in the same direction is performed.

At this time, since the yaw frame 207 connects the first handle 204, the rotation shaft 241, the rotation shaft 242, and the rotation shaft 243 to each other, the first handle 204, the yaw manipulation part 202, and the actuation manipulation part 203 are rotated together around the rotation shaft 243.

Next, the pitch motion is described as follows.

When a user rotates the first handle 204 around the rotation shaft 246 while holding the first handle 204, the actuation manipulation part 203, the yaw manipulation part 202, and the pitch manipulation part 201 are pitch-rotated around the rotation shaft 246. That is, when the pulley 210 of the first actuation manipulation part 251 to which the wires 301 and 305 are fixedly coupled is rotated around the rotation shaft 246, the wires 301 and 305 wound around the pulley 217 and the pulley 218 are moved. Similarly, when the pulley 220 of the second actuation manipulation part 256, to which the wires 302 and 306 are fixedly coupled, is rotated around the rotation shaft 246, the wires 302 and 306 wound around the pulley 227 and the pulley 228 are moved. At this time, as described with reference to FIGS. 9A and 9B, in order to allow the first jaw 101 and the second jaw 102 to pitch-rotate, the wires 301 and 305, which are first jaw wires, are moved in the same direction and respectively wound around the pulley 217 and the pulley 218, which are manipulation part pitch main pulleys, and the wires 302 and 306, which are second jaw wires, are moved in the same direction and respectively wound around the pulley 227 and the pulley 228, which are manipulation part pitch main pulleys. In addition, a rotating force is transmitted to the end tool 100 through the power transmission part 300, and two jaws 103 of the end tool 100 perform the pitch motion.

At this time, since the pitch frame 208 is connected to the yaw frame 207, and the yaw frame 207 connects the first handle 204, the rotation shaft 241, the rotation shaft 242, and the rotation shaft 243 to each other, when the pitch frame 208 is rotated around the rotation shaft 246, the yaw frame 207, the first handle 204, the rotation shaft 241, the rotation shaft 242, and the rotation shaft 243 connected to the pitch frame 208 are rotated together with the pitch frame 208. That is, when the pitch manipulation part 201 is rotated around the rotation shaft 246, the actuation manipulation part 203 and the yaw manipulation part 202 are rotated together with the pitch manipulation part 201.

In summary, in the surgical instrument 10 according to an embodiment of the present disclosure, the pulleys are formed on respective joint points (an actuation joint, a yaw joint, and a pitch joint), the wires (the first jaw wire or the second jaw wire) are wound around the pulleys, the rotational manipulations (actuation rotation, yaw rotation, and pitch rotation) of the manipulation part cause the movement of each wire, which in turn induces the desired motion of the end tool 100. Furthermore, the auxiliary pulley may be formed at one side of each of the pulleys, and the wire may not be wound several times around one pulley due to the auxiliary pulley.

FIG. 12 is a view schematically illustrating only a configuration of the pulleys and wires constituting joints of the surgical instrument 10 according to an embodiment of the present disclosure illustrated in FIG. 2. In FIG. 20, the relay pulleys that are not related to the operation of joints and are used to reroute the wire are omitted.

Referring to FIG. 12, the manipulation part 200 may include the pulley 210, the pulley 211, the pulley 212, the pulley 213, the pulley 214, the pulley 215, the pulley 216, the pulley 217, and the pulley 218 that are related to a rotational motion of the first jaw 101.

In addition, the manipulation part 200 may include the pulley 220, the pulley 221, the pulley 222, the pulley 223, the pulley 224, the pulley 225, the pulley 226, the pulley 227, and the pulley 228 that are related to a rotational motion of the second jaw 102 (the arrangement and structure of each of the pulleys of the manipulation part 200 are the same in principle as the arrangement and structure of each of the pulleys of the end tool 100, and thus specific designations of some reference numerals are omitted in the drawings).

The pulleys 211 and 212 and the pulleys 221 and 222 may be configured to be rotatable independently of each other around the same shaft, that is the rotation shaft 243. In this case, the pulleys 211 and 212 and the pulleys 221 and 222 may each be formed as two pulleys configured to face each other and configured to be independently rotatable.

The pulleys 213 and 214 and the pulleys 223 and 224 may be configured to be rotatable independently of each other around the same shaft, that is the rotation shaft 244. Here, the pulleys 213 and 214 may be formed as two pulleys configured to face each other and configured to be independently rotatable, and in this case, the two pulleys may be configured to have different diameters. Similarly, the pulleys 223 and 224 may be formed as two pulleys configured to face each other and configured to be independently rotatable, and in this case, the two pulleys may be configured to have different diameters.

The pulleys 215 and 216 and the pulleys 225 and 226 may be configured to be rotatable independently of each other around the same shaft, that is the rotation shaft 245. In this case, the pulleys 215 and 216 may be configured to have different diameters. In addition, the pulleys 225 and 226 may be configured to have different diameters.

The pulleys 217 and 218 and the pulleys 227 and 228 may be configured to be rotatable independently of each other around the same shaft, that is the rotation shaft 246.

The wire 301 is wound around the pulley 210 after sequentially passing through the pulley 217, the pulley 215, the pulley 213, and the pulley 211 of the manipulation part 200, and then is coupled to the pulley 210 by the coupling member 324. Meanwhile, the wire 305 sequentially passes through the pulley 218, the pulley 216, the pulley 214, and the pulley 212 of the manipulation part 200 and is coupled to the pulley 210 by the coupling member 324. Thus, when the pulley 210 is rotated, the wires 301 and 305 are wound around or unwound from the pulley 210, and accordingly, the first jaw 101 is rotated.

The wire 306 is wound around the pulley 220 after sequentially passing through the pulley 227, the pulley 225, the pulley 223, and the pulley 221 of the manipulation part 200, and then is coupled to the pulley 220 by the coupling member 327. Meanwhile, the wire 302 sequentially passes through the pulley 228, the pulley 226, the pulley 224, and the pulley 222 of the manipulation part 200 and is coupled to the pulley 220 by the coupling member 327. Thus, when the pulley 220 is rotated, the wire 302 and the wire 306 are wound around or unwound from the pulley 220, and accordingly, the second jaw 102 is rotated.

Thus, the actuation, yaw, and pitch manipulations can be manipulated independent of each

The actuation manipulation part 203, the yaw manipulation part 202, and the pitch manipulation part 201 are configured such that the respective rotation shafts are positioned at the rear thereof to be identical to the joint configuration of the end tool, so that a user can intuitively perform matching manipulations.

In particular, in the surgical instrument 10 according to an embodiment of the present disclosure, the pulleys are formed on respective joint points (an actuation joint, a yaw joint, and a pitch joint), the wires (the first jaw wire or the second jaw wire) are configured to be wound around the pulleys, the rotational manipulations (actuation rotation, yaw rotation, and pitch rotation) of the manipulation part cause the movement of each wire, which in turn induces the desired motion of the end tool 100. Furthermore, a first guide member 180 may be disposed on one side of the end tool jaw pulleys. Guide parts 183 and 184 serving as auxiliary pulleys may be formed on the first guide member 180. The guide parts 183 and 184 prevent the wires from winding multiple times around a single pulley, which ensures that the wires wound on the pulley do not come into contact with each other and that paths for the wires being wound around and unwound from the pulley are safely formed, thereby improving the safety and efficiency of power transmission through the wires.

Meanwhile, as described above, the yaw manipulation part 202 and the actuation manipulation part 203 are directly formed on the first handle 204. Thus, when the first handle 204 is rotated around the rotation shaft 246, the yaw manipulation part 202 and the actuation manipulation part 203 are also rotated together with the first handle 204. Accordingly, the coordinate systems of the yaw manipulation part 202 and the actuation manipulation part 203 are not fixed, but are continuously changed relative to the rotation of the first handle 204. That is, in FIG. 2 or the like, the yaw manipulation part 202 and the actuation manipulation part 203 are illustrated as being parallel to the z-axis. However, when the first handle 204 is rotated, the yaw manipulation part 202 and the actuation manipulation part 203 are not parallel to the Z-axis any longer. That is, the coordinate systems of the yaw manipulation part 202 and the actuation manipulation part 203 are changed according to the rotation of the first handle 204. However, in the present specification, for convenience of description, unless described otherwise, the coordinate systems of the yaw manipulation part 202 and the actuation manipulation part 203 are described on the basis of a state in which the first handle 204 is positioned perpendicular to the connection part 400 as illustrated in FIG. 2.

In the above, the operation of the end tool 100 of the surgical instrument 10 of the present disclosure has been described, focusing on the configuration of the wires and pulleys provided in the power transmission part 300 and the manipulation part 200. Meanwhile, the end tool 100 of the present disclosure can perform the above-described yaw, pitch, and actuation motions, while simultaneously receiving electrical energy from an external source to be used for procedures such as cauterizing or cutting body tissues. That is, the end tool 100 may be connected to an external power supply via the connector 410 of the manipulation part 200 to allow current to flow through at least a portion thereof to perform surgical operations. In particular, for the surgical instrument 10 corresponding to a bipolar electrosurgical instrument, two electrodes need to be formed on the end tool 100.

In the case of conventional bipolar electrosurgical instruments, each of the first and second jaws of the end tool was connected to a connector via electric wires to receive electrical energy supplied from an external power source. Conventionally, two electric wires, which are connected to the first and second jaws, passed through the connection part along with the plurality of wires used for performing the yaw, pitch, and actuation motions of the end tool and were connected to the end tool. As a result, there was an issue in which the volume and weight of the surgical instrument, including a coupling region between the end tool and the connection part, increased.

The present disclosure addresses these issues by providing a surgical instrument with a structure capable of forming two electrodes on the end tool, as described in various embodiments below.

### Electrode Formation Structure of End Tool

First, referring again to FIGS. 4 to 8 and the like, the end tool 100 may include one or more electric wires EC. The electric wires EC may extend from an external power supply (not shown) and receive electrical energy from the external power supply to transmit the electrical energy to the jaw 103. For example, the electric wires EC may be connected to the connector 410 of the manipulation part 200 described above to receive electrical energy from the external power supply. At this time, the electric wires EC may be provided as various types and forms of wires that allow current to flow.

The end tool 100 may receive electrical energy through the electric wire EC. In this case, the jaw 103 of the end tool 100 may be at least partially formed of a conductive material, thereby enabling the jaw 103 to receive the electrical energy to form an electrode.

The end tool 100 may include a coupling hub 150. The coupling hub 150 is positioned between the first and second jaws 101 and 102 and the connection part 400, and may transmit or insulate electrical energy to and from the first and second jaws 101 and 102.

The coupling hub 150 may be connected to one end of the connection part 400, and the first and second jaws 101 and 102 may be coupled to the coupling hub 150. That is, as described with reference to FIG. 4 and the like, the coupling hub 150, including the end tool hub 160 and the pitch hub 170, is positioned the first and second jaws 101 and 102 between the connection part 400, and refers to a member capable of accommodating pulleys, wires, and the like.

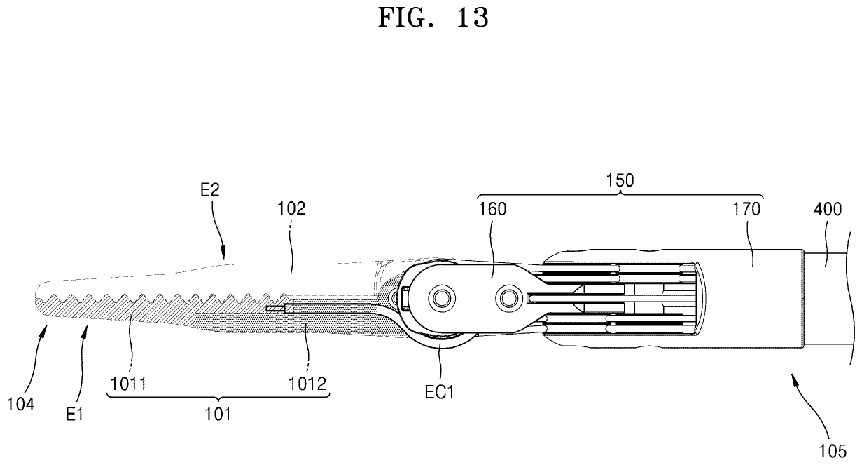

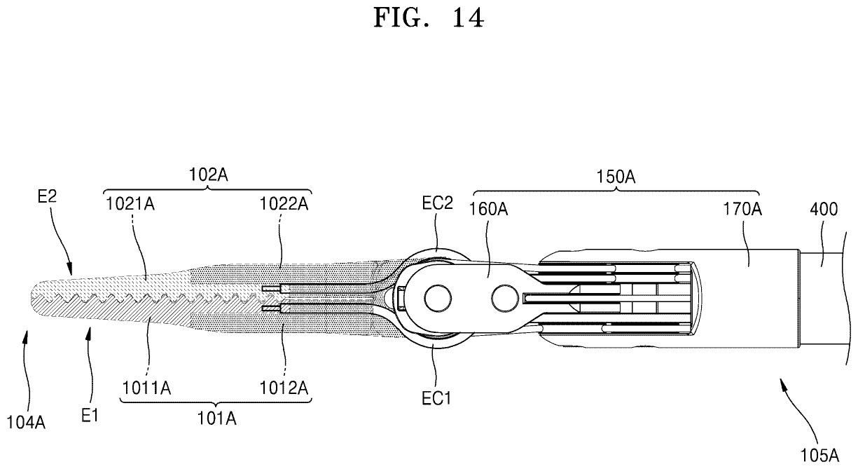

FIG. 13 is a side view illustrating an electrode formation structure of an end tool 100 according to an embodiment of the present disclosure. FIG. 14 is a side view illustrating an electrode formation structure of an end tool 100A according to another embodiment of the present disclosure.

Referring to FIGS. 13 and 14, a first jaw 101 or 101A and a second jaw 102 or 102A may receive electrical energy from an external power supply to form a first electrode E1 and a second electrode E2, respectively. Hereinafter, the principles by which the electrodes are formed will be described, focusing on the structure of an end tool 100 or 100A in each embodiment.

First, FIG. 13 illustrates an embodiment in which the coupling hub 150 includes a conductive material, the first jaw 101 receives electrical energy through the first electric wire EC1, and the second jaw 102 receives electrical energy through the coupling hub 150.

The first jaw 101 may include a first jaw electrode part 1011 and a first jaw insulating part 1012. The first jaw 101 may receive electrical energy through the first electric wire EC1 to form the first electrode E1.

The first jaw electrode part 1011 may receive electrical energy from an external power supply to form the first electrode E1.

Specifically, the first electric wire EC1 extending from the external power supply may be connected to the first jaw electrode part 1011. The first jaw electrode part 1011 may receive electrical energy through the first electric wire EC1 to form the first electrode E1. That is, the first jaw electrode part 1011 may be made of various conductive materials that allow current to flow therethrough.

The first jaw insulating part 1012 may be disposed between the first jaw electrode part 1011 and the coupling hub 150 to insulate the first jaw electrode part 1011. That is, the first jaw insulating part 1012 may be made of various insulating materials that do not allow current to flow therethrough.

In an embodiment, the first jaw electrode part 1011 is disposed on the end tool 100 at a distal end 104 side, the first jaw insulating part 1012 is disposed on the end tool 100 at a proximal end 105 side, and the first jaw insulating part 1012 may be disposed between the first jaw electrode part 1011 and the coupling hub 150. Accordingly, the first jaw insulating part 1012 is configured to prevent current from flowing between the first jaw electrode part 1011 and the coupling hub 150, thereby ensuring user safety.

In an embodiment, the first jaw electrode part 1011 and the first jaw insulating part 1012 may be formed by a double injection molding method. The first jaw electrode part 1011 and the first jaw insulating part 1012 may be formed using a double injection molding method by utilizing a conductive material and an insulating material, respectively, in a single mold. That is, rather than being provided as separate components to be assembled, the first jaw electrode part 1011 and the first jaw insulating part 1012 are integrally formed using a double injection molding method, thereby allowing the first jaw 101 to be formed with a reduced volume.

Meanwhile, the manufacturing method of the first jaw electrode part 1011 and the first jaw insulating part 1012 is not necessarily limited to the double injection molding method, and various methods, such as adhesion, coating, and the like, may also be used to integrally form the two regions of the first jaw 101.

The second jaw 102 may receive electrical energy through the coupling hub 150 to form the second electrode E2. The second jaw 102 may be at least partially in contact with the coupling hub 150, which is made of a conductive material, to receive electrical energy.

For example, as shown in FIG. 13, the second jaw 102 may be entirely formed of a conductive material and coupled to the coupling hub 150, which is made of a conductive material. The coupling hub 150 may be connected to an external power supply via a separate electric wire (not shown) or the like positioned in the connection part 400. Accordingly, when the coupling hub 150 receives electrical energy from an external power supply, current may flow through the coupling hub 150 and the second jaw 102, which are form of a conductive material. As a result, the second jaw 102 and the coupling hub 150 may together form the second electrode E2.

Specifically, the coupling hub 150 may include the end tool hub 160 and the pitch hub 170, as described above. Accordingly, the end tool hub 160 and the pitch hub 170 may each be at least partially formed of a conductive material, and portions thereof formed of the conductive material may be disposed to be in contact with each other. Accordingly, current can flow from the external power supply through the pitch hub 170, the end tool hub 160, and the second jaw 102, forming the second electrode E2 on the second jaw 102, the end tool hub 160, and the pitch hub 170.

Further, as described above, a plurality of pulleys may be disposed in the coupling hub 150 of the end tool 100. The plurality of pulleys are connected to the end tool 100, and may transmit a driving force to rotate the end tool 100. At this time, some of the plurality of pulleys may be formed of a conductive material, and may form the second electrode E2 together with the second jaw 102 and the coupling hub 150.

In an embodiment, at least one of the plurality of pulleys disposed in the coupling hub 150 may guide the first electric wire EC1 to the first jaw electrode part 1011. For example, as shown in FIG. 4, the first electric wire EC1 may be positioned along the pulley 111, the pulley 113, and the pulley 115 to be connected to the first jaw electrode part 1011. That is, the wire is positioned on the pulleys of the end tool 100 to transmit power, which enables the rotation of the end tool 100, and to guide the path of the first electric wire EC1, thereby allowing electrical energy to be transmitted to the first jaw electrode part 1011.

Up to this point, the embodiment has been described in which the first jaw 101 receives electrical energy through the electric wire EC, and the second jaw 102 receives electrical energy through the coupling hub 150, with reference to FIG. 13. Meanwhile, in the same manner as above, the first jaw 101 may receive electrical energy through the coupling hub 150, and the second jaw 102 may receive electrical energy through the electric wire EC. That is, the first jaw 101 may be formed of a conductive material along with the coupling hub 150, along the second jaw 102 may include a second jaw electrode part and a second jaw insulating part, with the second jaw electrode part connected to the electric wire EC.

The end tool 100 according to an embodiment of the present disclosure may include a conductive material only in a partial region of one of the first jaw 101 and the second jaw 102 to allow an electrode to be formed as current flows, while the remaining region is made of an insulating material to prevent accidents caused by current leakage and to enhance user safety.

Further, the end tool 100 according to an embodiment of the present disclosure may form an electrode by having another one of the first jaw 101 and the second jaw 102 formed of a conductive material along with the coupling hub 150 and connected to the coupling hub 150, thereby receiving electrical energy from an external power supply. That is, as another jaw receives electrical energy directly from the coupling hub 150 without a separate electric wire being directly connected to the interior thereof, the end tool 100 can be miniaturized and made lightweight.

Next, FIG. 14 illustrates an embodiment in which a coupling hub 150A is made of an insulating material, and the first jaw 101A and the second jaw 102A receive electrical energy respectively through a first electric wire EC1 and a second electric wire EC2.

The first jaw 101A includes a first jaw electrode part 1011A and a first jaw insulating part 1012A, and may receive electrical energy from an external power supply through the first electric wire EC1 to form a first electrode E1. As previously described with reference to FIG. 13, the first jaw electrode part 1011A includes a conductive material and is connected to the first electric wire EC1 to allow current to flow. In addition, the first jaw insulating part 1012A includes an insulating material and is disposed between the first jaw electrode part 1011A and the coupling hub 150A to prevent current from flowing therethrough.

In an embodiment, the first jaw electrode part 1011A and the first jaw insulating part 1012A may be formed by a double injection molding method. The first jaw electrode part 1011A and the first jaw insulating part 1012A may be formed using a double injection molding method by utilizing a conductive material and an insulating material, respectively, in a single mold. That is, rather than being provided as separate components to be assembled, the first jaw electrode part 1011A and the first jaw insulating part 1012A are integrally formed using a double injection molding method, thereby allowing the first jaw 101A to be manufactured with a reduced volume.

Meanwhile, the manufacturing method of the first jaw electrode part 1011A and the first jaw insulating part 1012A is not necessarily limited to the double injection molding method, and various methods, such as adhesion, coating, and the like, may also be used to integrally form the two regions of the first jaw 101A.

The second jaw 102A includes a second jaw electrode part 1021A and a second jaw insulating part 1022A, and may receive electrical energy from an external power supply through the second electric wire EC2 to form a second electrode E2.

Specifically, the second electric wire EC2 extending from the external power supply may be connected to the second jaw electrode part 1021A. The second jaw electrode part 1021A may receive electrical energy through the second electric wire EC2 to form the second electrode E2. That is, the second jaw electrode part 1021A may include various conductive materials that allow current to flow therethrough. Similar to the first electric wire EC1, which is connected to the first jaw 101A, the second electric wire EC2 may be positioned along the pulleys in the coupling hub 150A and guided to the second jaw electrode part 1021A.

The second jaw insulating part 1022A may be disposed between the second jaw electrode part 1021A and the coupling hub 150A to insulate the second jaw electrode part 1021A. That is, the second jaw insulating part 1022A may include various insulating materials that do not allow current to flow therethrough.

In an embodiment, the second jaw electrode part 1021A is disposed on the end tool 100A at a distal end 104A side, the second jaw insulating part 1022A is disposed on the end tool 100A at a proximal end 105A side, and the second jaw insulating part 1022A may be disposed between the second jaw electrode part 1021A and the coupling hub 150A. That is, the second jaw electrode part 1021A may face the first jaw electrode part 1011A. Through this configuration, the first electrode E1 and the second electrode E2, which face each other, may be formed in the end tool 100A, and the second jaw insulating part 1022A is configured to prevent current from flowing between the second jaw electrode part 1021A and the coupling hub 150A, thereby ensuring user safety.

In an embodiment, the second jaw electrode part 1021A and the second jaw insulating part 1022A may be formed by a double injection molding method. The second jaw electrode part 1021A and the second jaw insulating part 1022A may be formed using a double injection molding method that utilizes both conductive and insulating materials within a single mold. That is, rather than being provided as separate components to be assembled, the second jaw electrode part 1021A and the second jaw insulating part 1022A are integrally formed using a double injection molding method, thereby allowing the second jaw 102A to be manufactured with a reduced volume.

Meanwhile, the manufacturing method of the second jaw electrode part 1021A and the second jaw insulating part 1022A is not necessarily limited to the double injection molding method, and various methods, such as adhesion, coating, and the like, may also be used to integrally form the two regions of the second jaw 102A.

The end tool 100A according to another embodiment of the present disclosure may have the first jaw 101A and the second jaw 102A, each provided with a conductive material only in a partial region, thereby enabling current to flow and forming the first electrode E1 and the second electrode E2. The first jaw 101A and the second jaw 102A have regions formed of a conductive material, which are respectively connected to the first electric wire EC1 and the second electric wire EC2 to receive electrical energy. In addition, the remaining regions of the first jaw 101A and the second jaw 102A, as well as the coupling hub 150A, are made of an insulating material to prevent accidents caused by current leakage and to enhance user safety. In addition, since the electrode part and the insulating part of each of the first jaw 101A and the second jaw 102A are integrally formed using a double injection molding method or the like, the end tool 100A can be miniaturized and made lightweight, even when the first electric wire EC1 and the second electric wire EC2 are connected to the first jaw 101A and the second jaw 102A, respectively.

Hereinafter, another end tool 500 applicable to the surgical instrument 10 of the present disclosure will be described, with a focus on the electrode formation structure of the end tool 500.

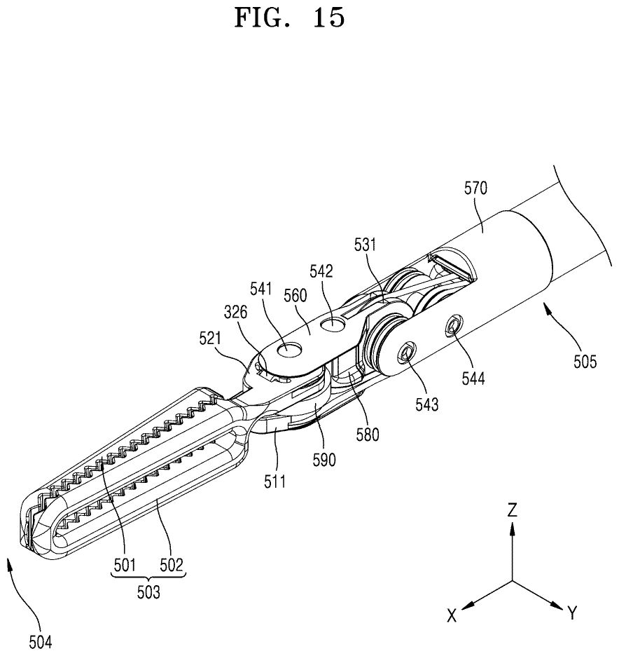

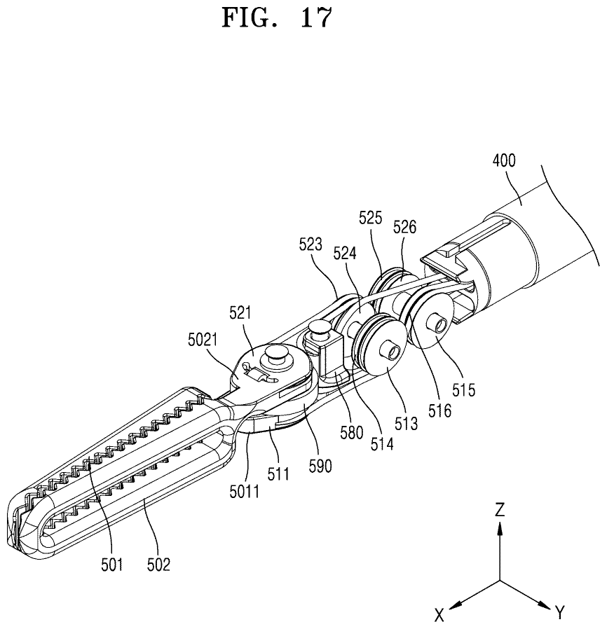

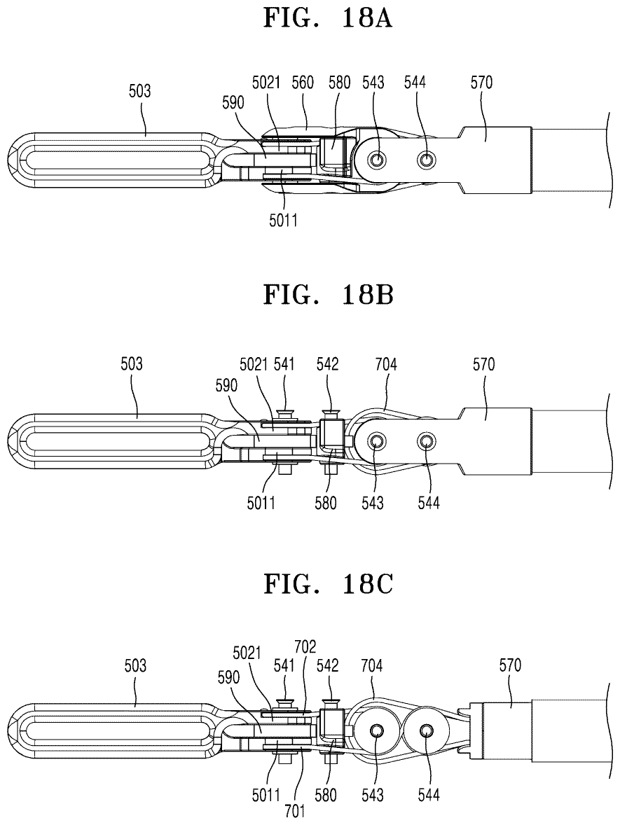

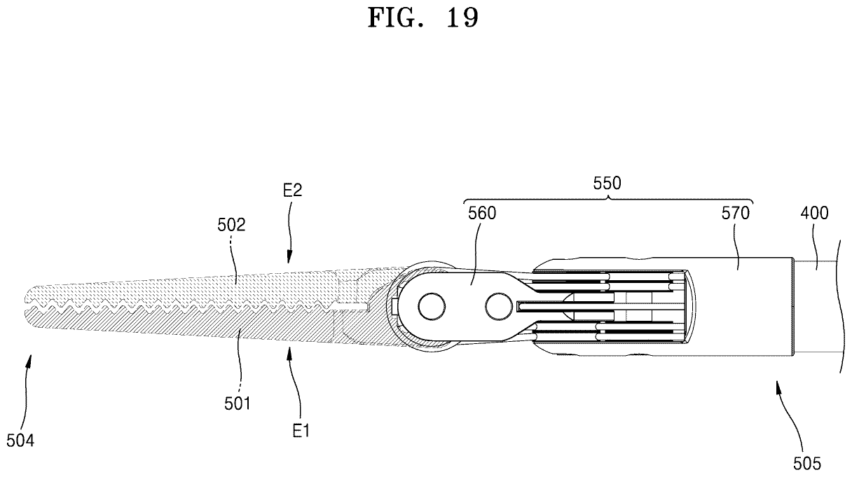

FIGS. 15 to 17 are perspective views illustrating an end tool 500 of a surgical instrument 10 according to another embodiment of the present disclosure. FIGS. 18A-18C are side views of the end tool 500 of FIG. 15, and FIG. 19 is a plan view illustrating an electrode formation structure of the end tool 500 of FIG. 15.

The surgical instrument 10 of FIG. 15 features a wire configuration of the end tool 500 and a power transmission part 700, as well as the principle of electrode formation of the end tool 500 based on this configuration, and thus, the following description will focus on these features. For other configurations, reference will be made to the descriptions provided above with reference to FIGS. 4 to 12, regarding the connection part 400, the power transmission part 300, the manipulation part 200, and the like of the surgical instrument 10.

Referring to FIGS. 15 to 19, the end tool 500 may receive power through wires 701, 702, 703, 704, 705, and 706 of the power transmission part 700 and rotate. Here, the wire 701 and the wire 705 may be paired to serve as first jaw wires. The wire 702 and the wire 706 may be paired to serve as second jaw wires. Here, the components encompassing the wire 701 and the wire 705, which are first jaw wires, and the wire 702 and the wire 706, which are second jaw wires, may be referred to as jaw wires. In addition, the wire 703 and the wire 704 may be paired to serve as pitch wires. The operation of the end tool 500 according to the driving of the wires 701, 702, 703, 704, 705, and 706 is referenced from the descriptions provided above with reference to FIGS. 4 to 12.

The end tool 500 may receive electrical energy through the jaw wires 701, 702, 705, and 706. A first jaw 501 and a second jaw 502 are connected to first jaw wires 701 and 705 and second jaw wires 702 and 706, respectively, to receive electrical energy and form a first electrode E1 and a second electrode E2.

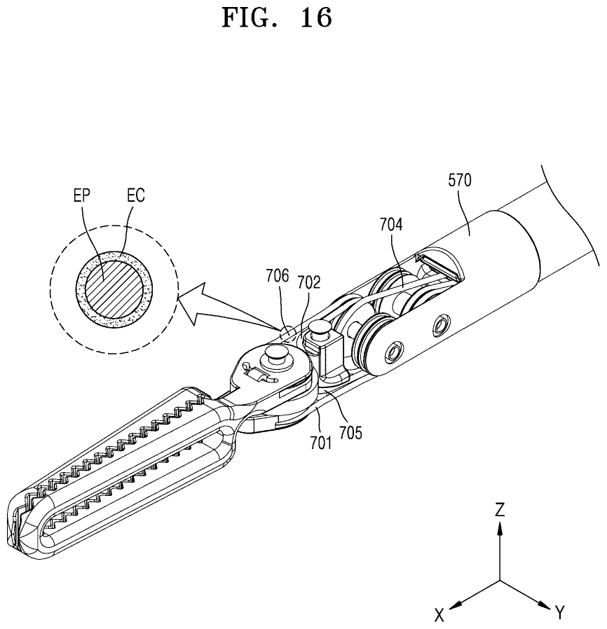

As shown in FIG. 16, the jaw wires 701, 702, 705, and 706 may be provided with an electric wire part EP and a cover part CP. The jaw wires 701, 702, 705, and 706 may be partially formed of a conductive material and may transmit electrical energy from an external power supply to the end tool 500.

The electric wire part EP may be connected to the external power supply and may be formed of a conductive material. The jaw wires 701, 702, 705, and 706 may allow current to flow through the electric wire part EP and transmit the electrical energy to the end tool 500.

The cover part CP may surround the electric wire part EP and may be formed of an insulating material. When current flows through the electric wire part EP of the jaw wires 701, 702, 705, and 706, the cover part CP provides electrical insulation from the outside, thereby ensuring user safety.

In an embodiment, the cover part CP may be formed as a sheath surrounding the electric wire part EP. The cover part CP may be formed as a sheath by coating the electric wire part EP with an insulating material.

In another embodiment, the cover part CP may be formed as a tube coupled to the electric wire part EP. The cover part CP may be formed as a tube made of an insulating material and coupled to the electric wire part EP. For example, the cover part CP may be provided as a Teflon-based or insulating shrink tube and may be coupled to the electric wire part EP.

As such, by providing the jaw wires 701, 702, 705, and 706 with the electric wire part EP, which allows current to flow, and the cover part CP, which surrounds and insulates the electric wire part EP from the outside, electrical energy can be transmitted to the jaws 501 and 502 while simultaneously preventing current leakage or the like. That is, the jaw wires 701, 702, 705, and 706 can simultaneously serve as driving wires for controlling the operation of the end tool 500 and as electric wires for transmitting electrical energy to the end tool 500.

The first jaw 501 may be include a first wire-contact part 5011. The first wire-contact part 5011 is coupled to the coupling hub 550, and may be in contact with current-carrying portions of the first jaw wires 701 and 705, i.e., a first electrode part EP1. At this time, the first wire-contact part 5011 may be integrally formed with a pulley 511 that functions as an end tool first jaw pulley, as shown in FIG. 15.

At least a portion of the first jaw 501 may be formed of a conductive material. For example, the first wire-contact part 5011 is formed of a conductive material to allow current received through the first jaw wires 701 and 705 to flow. The first jaw 501 may include at least a partial region, including the first wire-contact part 5011, formed of a conductive material, thereby forming the first electrode E1 when supplied with electrical energy.

The second jaw 502 may include a second wire-contact part 5021. The second wire-contact part 5021 is coupled to the coupling hub 550, and may be in contact with current-carrying portions of the second jaw wires 702 and 706, i.e., a second electrode part EP2. At this time, the second wire-contact part 5021 may be integrally formed with a pulley 521 that functions as an end tool second jaw pulley, as shown in FIG. 15.

At least a portion of the second jaw 502 may be formed of a conductive material. For example, the second wire-contact part 5021 may be formed of a conductive material to allow current received from the second jaw wires 702 and 706 to flow. The second jaw 502 may include at least a partial region, including the second wire-contact part 5021, formed of a conductive material, thereby forming the second electrode E2 when supplied with electrical energy.

As described above, the first jaw 501 and the second jaw 502 may include a conductive material to form electrodes, and may be in contact with the first electric wire EC1 and the second electric wire EC2, respectively, in the coupling hub 550 to receive electrical energy. In other words, since the electric wires EC1 and EC2 are positioned not to be connected inside the first jaw 501 and the second jaw 502 but instead to be in contact with the first wire-contact part 5011 and the second wire-contact part 5021 within the coupling hub 550, the first jaw 501 and the second jaw 502 can be miniaturized.

The end tool 500 may further include an insulating sleeve IS. The insulating sleeve IS is made of an insulating material and may be disposed between the first wire-contact part 5011 and the second wire-contact part 5021. In addition, the insulating sleeve IS may be disposed between the first wire-contact part 5011 and the coupling hub 550, or between the second wire-contact part 5021 and the coupling hub 550. By insulating the first electrode E1 formed on the first jaw 501 and the second electrode E2 formed on the second jaw 502 in the end tool 500 with the insulating sleeve IS, the tissue can be positioned between the two electrodes and can be stably cauterized and cut.

The end tool 500 according to another embodiment of the present disclosure may receive electrical energy as the first and second jaws 501 and 502 come into contact with the jaw wires 701, 702, 705, and 706, which include the electric wire part EP and the cover part CP. That is, the jaw wires 701, 702, 705, and 706, made of a conductive material, can rotate the first jaw 501 and the second jaw 502 while simultaneously supplying electrical energy to form two electrodes in the end tool 500. In addition, the jaw wires 701, 702, 705, and 706 may come into contact with the first wire-contact part 5011 of the first jaw 501 and the second wire-contact part 5021 of the second jaw 502 in the coupling hub 550. This allows the first jaw 501 and the second jaw 502 to receive electrical energy through the jaw wires 701, 702, 705, and 706 in the coupling hub 550, even without separate wires extending into the interiors of the jaws 501 and 502, thereby enabling the end tool 500 to be miniaturized and made lightweight.

As described above, in the surgical instrument of the present disclosure, the coupling hub of the end tool includes a conductive material and is connected to an external power supply. One of the pair of jaws can receive electrical energy directly from the coupling hub, while another one thereof can receive electrical energy through the electric wire. This configuration allows only one electric wire to be connected inside the jaw, while another electric wire is connected to the coupling hub from the connection part, thereby enabling the end tool to be miniaturized and made lightweight.

Alternatively, in the surgical instrument of the present disclosure, the coupling hub of the end tool may be formed of an insulating material, and the pair of jaws may receive electrical energy through the respective electric wires. In this case, each jaw may be integrally and compactly formed using methods such as double injection molding, coating, or adhesion.

Alternatively, in the surgical instrument of the present disclosure, the jaw wires may be made a conductive material and may include an electric wire part and a cover part, enabling the jaw wires to control the rotational motion of the end tool while simultaneously transmitting electrical energy to the end tool. In this case, the jaw wires may come into contact with the pair of jaws inside the coupling hub, so that electrodes can be formed on the jaws without the jaw wires being connected inside the jaws, thereby enabling the end tool to be miniaturized and made lightweight.

In a surgical instrument according to an embodiment of the present disclosure, an end tool includes a coupling hub made of a conductive material, thereby allowing one of a pair of jaws to receive electrical energy directly from the coupling hub, while another one thereof receives electrical energy through an electric wire. Alternatively, the coupling hub of the end tool can be formed of an insulating material, thereby allowing each of the pair of jaws to receive electrical energy through the respective electric wires, with each jaw formed using a double injection molding method. Accordingly, a volume and weight of the end tool can be reduced, and user convenience and stability can be improved.

In a surgical instrument according to an embodiment of the present disclosure, jaw wires configured to control a rotational motion of an end tool can include a conductive material and transmit electrical energy to a pair of jaws. In this case, the jaw wires can come into contact with the pair of jaws, respectively, in a coupling hub, thereby enabling the end tool to be miniaturized and made lightweight.

The present disclosure has been described above with a focus on exemplary embodiments. It will be understood by those skilled in the art that various changes in form and details may be made therein without departing from the essential features of the present disclosure. Therefore, the disclosed embodiments should be considered in descriptive sense only and not for purposes of limitation. The scope of the present disclosure is defined not by the detailed description of the disclosure but by the appended claims, and all differences within the scope will be construed as being included in the present disclosure.

What is claimed is:

1. A surgical instrument comprising:
an end tool rotatable in at least one direction and including a first jaw and a second jaw opposite to the first jaw;
a manipulation part configured to control a rotational motion of the end tool, connected to an external power supply, and further configured to transmit an electrical energy provided by the external power supply to the end tool; and
a connection part configured to connect the end tool to the manipulation part,
wherein the end tool further includes a coupling hub connected to one end of the connection part, and
the first jaw includes a first jaw electrode part electrically connected to the external power supply and configured to receive the electrical energy, and a first jaw insulating part disposed between the first jaw electrode part and the coupling hub and configured to insulate the first jaw electrode part.

2. The surgical instrument of claim 1, wherein
the coupling hub is electrically connected to the external power supply and receives the electrical energy, and
the second jaw receives the electrical energy through the coupling hub.

3. The surgical instrument of claim 2, wherein at least a portion of the second jaw, which is in contact with the coupling hub, is formed of a conductive material.

4. The surgical instrument of claim 3, wherein the coupling hub includes an end tool hub coupled to one end of the end tool, and a pitch hub configured to connect the end tool hub to the connection part and disposed to at least partially overlap the end tool hub,
wherein at least one of the end tool hub and the pitch hub comprises conductive material.

5. The surgical instrument of claim 2, wherein the end tool further includes a plurality of pulleys disposed on the coupling hub and configured to transmit power for rotating the end tool,
wherein some of the plurality of pulleys include a conductive material and are electrically connected to the external power supply.

6. The surgical instrument of claim 5, wherein at least one of the plurality of pulleys guides, to the first jaw electrode part, a first electric wire which is electrically connected to the external power supply and through which the electrical energy is transmitted to the first jaw electrode part.

7. The surgical instrument of claim 1, wherein the second jaw includes a second jaw electrode part electrically connected to the external power supply and configured to receive the electrical energy, and a second jaw insulating part disposed between the second jaw electrode part and the coupling hub and configured to insulate the second jaw electrode part.

8. The surgical instrument of claim 7, wherein the coupling hub is formed of an insulating material.

9. The surgical instrument of claim 7, wherein
the first jaw electrode part and the first jaw insulating part are formed using a double injection molding method, and
the second jaw electrode part and the second jaw insulating part are formed using the double injection molding method.

10. The surgical instrument of claim 7, wherein the end tool further includes a plurality of pulleys disposed on the coupling hub and configured to transmit power for rotating the end tool,
wherein at least one of the plurality of pulleys guides, to the first jaw electrode part, a first electric wire which is electrically connected to the external power supply and through which the electrical energy is transmitted to the first jaw electrode part, and guides, to the second jaw electrode part, a second electric wire which is electrically connected to the external power supply and through which the electrical energy is transmitted to the second jaw electrode part.

11. A surgical instrument comprising:
an end tool rotatable in at least one direction and including a first jaw and a second jaw opposite to the first jaw;
a manipulation part configured to control a rotational motion of the end tool, connected to an external power supply, and further configured to transmit an electrical energy provided by the external power supply to the end tool;
a connection part configured to connect the end tool to the manipulation part; and
a power transmission part connected to the manipulation part and including a jaw wire configured to transmit a rotation of the manipulation part to the end tool,
wherein the end tool further includes a coupling hub connected to one end of the connection part, and
the jaw wire includes a first jaw wire positioned on the coupling hub and electrically connected to the first jaw, and a second jaw wire positioned on the coupling hub and electrically connected to the second jaw.

12. The surgical instrument of claim 11, wherein
the first jaw includes a first wire-contact part that is coupled to the coupling hub and in contact with a portion of a first electric wire part of the first jaw wire, and
the second jaw includes a second wire-contact part that is coupled to the coupling hub and in contact with a portion of a second electric wire part of the second jaw wire.

13. The surgical instrument of claim 12, wherein the end tool further includes an insulating sleeve disposed between the first wire-contact part and the second wire-contact part, and formed of an insulating material.

14. The surgical instrument of claim 12, wherein the coupling hub is formed of an insulating material.

15. The surgical instrument of claim 11, wherein the jaw wire includes an electric wire part connected to the external power supply and formed of a conductive material, and a cover part surrounding the electric wire part and formed of an insulating material.

16. The surgical instrument of claim 15, wherein the cover part is formed as a sheath surrounding the electric wire part.

17. The surgical instrument of claim 15, wherein the cover part is formed as a tube coupled to the electric wire part.
